SEQUENCE LISTING (83 sequences)

SEQ ID NO: 1 - protein, 268 aa, Artificial
  synthetic sequence
  MISC_FEATURE (2)..(2): X at position 2 is V or A
  MISC_FEATURE (3)..(3): X at position 3 is K or null
  MISC_FEATURE (4)..(4): X at position 4 is S or null
  MISC_FEATURE (5)..(5): X at position 5 is N or H
  MISC_FEATURE (6)..(6): X at position 6 is I, V, L or M
  MISC_FEATURE (8)..(8): X at position 8 is S, G, Y or C
  MISC_FEATURE (10)..(10): X at position 10 is I, L or M
  MISC_FEATURE (12)..(12): X at position 12 is V, A, or L
  MISC_FEATURE (16)..(16): X at position 16 is A, T, or V
  MISC_FEATURE (17)..(17): X at position 17 is T or M
  MISC_FEATURE (19)..(19): X at position 19 is S or T
  MISC_FEATURE (21)..(21): X at position 21 is V, I, or M
  MISC_FEATURE (23)..(23): X at position 23 is L or F
  MISC_FEATURE (27)..(27): X at position 27 is R or W
  MISC_FEATURE (33)..(33): X at position 33 is G or null
  MISC_FEATURE (39)..(39): X at position 39 is G or null
  MISC_FEATURE (47)..(47): X at position 47 is S or I
  MISC_FEATURE (55)..(55): X at position 55 is P or S
  MISC_FEATURE (59)..(59): X at position 59 is G or null
  MISC_FEATURE (60)..(60): X at position 60 is G or T
  MISC_FEATURE (76)..(76): X at position 76 is G or S
  MISC_FEATURE (84)..(84): X at position 84 is G or S
  MISC_FEATURE (92)..(92): X at position 92 is G or null
  MISC_FEATURE (93)..(93): X at position 93 is W or null
  MISC_FEATURE (94)..(94): X at position 94 is G or null
  MISC_FEATURE (95)..(95): X at position 95 is Q or null
  MISC_FEATURE (96)..(96): X at position 96 is P or null
  MISC_FEATURE (97)..(97): X at position 97 is H or null
  MISC_FEATURE (98)..(98): X at position 98 is G or null
  MISC_FEATURE (99)..(99): X at position 99 is G or null
  MISC_FEATURE (100)..(100): X at position 100 is G or null
  MISC_FEATURE (105)..(105): X at position 105 is G or S
  MISC_FEATURE (107)..(107): X at position 107 is G or null
  MISC_FEATURE (108)..(108): X at position 108 is T, A, or S
  MISC_FEATURE (110)..(110): X at position 110 is G, N, or S
  MISC_FEATURE (113)..(113): X at position 113 is N or G
  MISC_FEATURE (116)..(116): X at position 116 is S or N
  MISC_FEATURE (121)..(121): X at position 121 is N or S
  MISC_FEATURE (122)..(122): X at position 122 is M or L
  MISC_FEATURE (125)..(125): X at position 125 is V or M
  MISC_FEATURE (142)..(142): X at position 142 is M or L
  MISC_FEATURE (151)..(151): X at position 151 is L, M or I
  MISC_FEATURE (156)..(156): X at position 156 is N or S
  MISC_FEATURE (158)..(158): X at position 158 is Y or W
  MISC_FEATURE (168)..(168): X at position 168 is Y or H
  MISC_FEATURE (172)..(172): X at position 172 is N or E
  MISC_FEATURE (177)..(177): X at position 177 is R or K
  MISC_FEATURE (179)..(179): X at position 179 is V or M
  MISC_FEATURE (180)..(180): X at position 180 is D, N or S
  MISC_FEATURE (181)..(181): X at position 181 is Q, R or E
  MISC_FEATURE (183)..(183): X at position 183 is S or N
  MISC_FEATURE (184)..(184): X at position 184 is S or N
  MISC_FEATURE (187)..(187): X at position 187 is N, S, or T
  MISC_FEATURE (190)..(190): X at position 190 is H or R
  MISC_FEATURE (197)..(197): X at position 197 is V or I
  MISC_FEATURE (199)..(199): X at position 199 is Q or E
  MISC_FEATURE (216)..(216): X at position 216 is V, M or I
  MISC_FEATURE (218)..(218): X at position 218 is M or I
  MISC_FEATURE (221)..(221): X at position 221 is R or Q
  MISC_FEATURE (228)..(228): X at position 228 is I or V
  MISC_FEATURE (232)..(232): X at position 232 is Q, E, or R
  MISC_FEATURE (233)..(233): X at position 233 is R, Q, or K
  MISC_FEATURE (235)..(235): X at position 235 is S or Y
  MISC_FEATURE (236)..(236): X at position 236 is Q or E
  MISC_FEATURE (238)..(238): X at position 238 is Y, S, F, or A
  MISC_FEATURE (239)..(239): X at position 239 is Y or Q
  MISC_FEATURE (240)..(240): X at position 240 is Q, D, or G
  MISC_FEATURE (241)..(241): X at position 241 is G or null
  MISC_FEATURE (242)..(242): X at position 242 is R or null
  MISC_FEATURE (243)..(243): X at position 243 is R or K
  MISC_FEATURE (244)..(244): X at position 244 is G, S, or A
  MISC_FEATURE (245)..(245): X at position 245 is A or S
  MISC_FEATURE (246)..(246): X at position 246 is S or G
  MISC_FEATURE (247)..(247): X at position 247 is V, T, A, M or null
  MISC_FEATURE (248)..(248): X at position 248 is I, L, or V
  MISC_FEATURE (249)..(249): X at position 249 is L or I
  MISC_FEATURE (252)..(252): X at position 252 is S or P
  MISC_FEATURE (256)..(256): X at position 256 is I or V
  MISC_FEATURE (261)..(261): X at position 261 is F or L
  MISC_FEATURE (264)..(264): X at position 264 is F or L
  MISC_FEATURE (268)..(268): X at position 268 is G or R
MXXXXXGXWXLXLFVXXWXDXGXCKKXPKPGGXWNTGGXSRYPGQGXPGGNRYPXQGGXXWGQPHGGGWGQPHGGXWGQPHGGXWGQPHGGXXXXXXXXXGWGQXGXXHXQWXKPXKPKTXXKHXAGAAAAGAVVGGLGGYXLGSAMSRPXIHFGXDXEDRYYRENMXRYPXQVYYXPXXXYXXQNXFVXDCVNITXKXHTVTTTTKGENFTETDXKXMEXVVEQMCXTQYXXEXXAXXXXXXXXXXXXFSXPPVXLLISXLIXLIVX

SEQ ID NO: 2 - protein, 257 aa, dog
MVKSHIGGWILLLFVATWSDVGLCKKRPKPGGWNTGGGSRYPGQGSPGGNRYPPQGGGGWGQPHGGGWGQPHGGGWGQPHGGGWGQPHGGGGWGQGGGSHSQWGKPNKPKTNMKHVAGAAAAGAVVGGLGGYMLGSAMSRPLIHFGNDYEDRYYRENMYRYPEQVYYRPVDQYSNQNNFVRDCVNITVKQHTVTTTTKGENFTETDMKIMERVVEQMCVTQYQKESEAYYQRGASAILFSPPPVILLISLLILLIVG

SEQ ID NO: 3 - protein, 255 aa, horse
MVKSHVGGWILVLFVATWSDVGLCKKRPKPGGWNTGGSRYPGQGSPGGNRYPPQGGGGWGQPHGGGWGQPHGGGWGQPHGGGWGQPHGGGGWGQGGSHGQWNKPSKPKTNMKHVAGAAAAGAVVGGLGGYMLGSAMSRPLIHFGNDYEDRYYRENMYRYPNQVYYRPVSEYSNQNNFVHDCVNITVKQHTVTTTTKGENFTETDVKIMERVVEQMCITQYQKEYEAFQQRGASVVLFSSPPVVLLISFLIFLIVG

SEQ ID NO: 4 - protein, 257 aa, mink
MVKSHIGSWLLVLFVATWSDIGFCKKWPKPGGGWNTGGSRYPGQGSPGGNRYPPQGGGGWGQPHGGGWGQPHGGGWGQPHGGGWGQPHGGGGWGQGGGSHGQWGKPSKPKTNMKHVAGAAAAGAVVGGLGGYMLGSAMSRPLIHFGNDYEDRYYRENMYRYPNQVYYKPVDQYSNQNNFVHDCVNITVKQHTVTTTTKGENFTETDMKIMERVVEQMCVTQYQRESEAYYQKGASAILFSPPPVILLISLLILLIVG

SEQ ID NO: 5 - protein, 254 aa, mouse
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 6 - protein, 256 aa, sheep
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDRYSNQNNFVHDCVNITVKQHTVTTTTKGENFTETDIKIMERVVEQMCITQYQRESQAYYQRGASVILFSSPPVILLISFLIFLIVG

SEQ ID NO: 7 - protein, 256 aa, red deer
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 8 - protein, 256 aa, deer
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQSGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVG

SEQ ID NO: 9 - protein, 256 aa, deer
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVG

SEQ ID NO: 10 - protein, 256 aa, deer
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGAHSQWNKPSKPKTNMKHVAGAAAAGAVVGGLGGYMLGSAMSRPLIHFGNDYEDRYYRENMYRYPNQVYYRPVDQYNNQNTFVHDCVNITVKQHTVTTTTKGENFTETDIKMMERVVEQMCITQYQRESQAYYQRGASVILFSSPPVILLISFLIFLIVR

SEQ ID NO: 11 - protein, 256 aa, elk
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYLLGSAMSRPLIHFGNDYEDRYYRENMYRYPNQVYYRPVDQYNNQNTFVHDCVNITVKQHTVTTTTKGENFTETDIKMMERVVEQMCITQYQRESEAYYQRGASVILFSSPPVILLISFLIFLIVG

SEQ ID NO: 12 - protein, 202 aa, Nubian ibex
KPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQ

SEQ ID NO: 13 - protein, 256 aa, muskox
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG

SEQ ID NO: 14 - protein, 227 aa, nyala
MWSDVGLCKKRPKPGGGWNTGGSRYPGQGSPGGNRYPSQGGGGWGQPHGGGWGQPHGGGWGQPHGGGWGQPHGGGGWGQGGTHGQWNKPSKPKTNMKHVAGAAAAGAVVGGLGGYMLGSAMSRPLTHFGNDYEDHYYRENMYRYPNQVYYRPVDQYSNQNNFVHDCVNITVKQHTVTTTTKGENFTETDIKMMERVVEQMCITQYRRESQAYYQRGASVILFSSPPV

SEQ ID NO: 15 - protein, 223 aa, sable antelope
PKPGGGWNTGGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFL

SEQ ID NO: 16 - protein, 195 aa, Addax
GGSRYPGQGSPGGNRYPPQGGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQR

SEQ ID NO: 17 - protein, 215 aa, banteng
PKPGGGWNTGGSRYPGQGSPGGNRYPPQGGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNSFVHDCVNITVKEHTVTTTTKGENFTETDIKMMERVVEQMCITQYRRESQAYYQRGASVILFSSPP

SEQ ID NO: 18 - protein, 226 aa, goitered gazelle
GGGWNTGGSRYPGQGSPGGNRYPPQGGGGWGQPHGGGWGQPHGGGWGQPHGGGWGQPHGGGWGQPHGGGGWGQGGTHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKMMERVVEQMCITQYRRESQAYYQRGASVILFSSPPVILLIS

SEQ ID NO: 19 - protein, 256 aa, Capra hircus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMEQVVEQMCITQYQRESQAYYQRGASVILFSSPPVILLISFLIFLIVG

SEQ ID NO: 20 - protein, 256 aa, Capra hircus; identical to SEQ ID NO: 13

SEQ ID NO: 21 - protein, 181 aa, takin
PKPGGGWNTGGSRYPGQGSPGGNRYPPQGGGGWGQPHGGGWGQPHGGGWGQPHGGGWGQPHGGGGWGQGGSHSQWNKPSKPKTNMKHVAGAAAAGAVVGGLGGYMLGSAMSRPLIHFGSDYEDRYYRENMYRYPNQVYYRPVDQYSNQNNFVHDCVNITVKQHTVTTTTKGENFTETDIKI

SEQ ID NO: 22 - protein, 216 aa, Cow
YPGQGIPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGNDYEDRYYRENMHRYPNQVYYRPVDQYSNQNNFVHDCVNITVKEHTVTTTTKGENFTETDIKMMERVVEQMCITQYRRESQAYYQRGASVILFSSPPVILLIS

SEQ ID NO: 23 - protein, 263 aa, water buffalo
MVKSHIGSWILVLFVVMWSDVGLCKKRPKPGGGWNTGGSRYPGQGSPGGNRYPSQGGGGWGQPHGGGWGQPHGGGWGQPHGGGWGQPHGGGWGQPHGGGGWGQGGTHGQWNKPSKPKTMKHMAGAAAAGAVVGGLGGYMLGSAMSRPLIHFGNDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISLLIFLIVR

SEQ ID NO: 24 - protein, 264 aa, water buffalo
MVKSHIGSWILVLFVVMWSDVGLCKKRPKPGGGWNTGGSRYPGQGSPGGNRYPSQGGGGWGQPHGGGWGQPHGGGWGQPHGGGWGQPHGGGWGQPHGGGGWGQGGTHSQWNKPSKPKTNMKHMAGAAAAGAVVGGLGGYMLGSAMSRPLIHFGNDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISLLIFLIVR

SEQ ID NO: 25 - protein, 200 aa, European bison
QGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCITQYQRESQAYYQRGASV

SEQ ID NO: 26 - protein, 252 aa, Xiji Donkey
MVKSHVGWILVLFVTWSDVGLCKKRPKPGGWNTGGSRYPGQGSPGGNRYPPQGGGGWGQPHGGGWGQPHGGGWGQPHGGGWGQPHGGGWGQGGSHGQWNKPSKPKTNMKHVAGAAAAGAVVGGLGGYMLGSAMSRPLIHFGNDYEDRYYRENMYRYPNQVYYRPVNEYSNQNNFVHDCVNITVKQHTVTTTTKGENFTETDVKIMERVVEQMCITQYQKEYEAFQQRGASVVLFSSPPVVLLISFLIFLIVG

SEQ ID NO: 27 - protein, 255 aa, camelus
MVKSHMGSWILVLFVVTWSDVGLCKKRPKPGGGWNTGGSRYPGQGSPGGNRYPPQGGGGWGQPHGGGWGQPHGGGWGQPHGGGWGQPHGGGWGQGGGAHGQWNKPSKPKTSMKHVAGAAAAGAVVGGLGGYMLGSAMSRPLIHFGNDYEDRYYRENMYRYPNQVYYKPVDQYSNQNSFVHDCVNITVKQHTVTTTTKGENFTETDVKMMERVVEQMCITQYQREYQASYGRGASVIFSSPPVILLISFLIFLIVG

SEQ ID NO: 28 - protein, 255 aa, alpaca
MVKSHMGSWILVLFVVTWSDVGLCKKRPKPGGGWNTGGSRYPGQGSPGGNRYPPQGGGGWGQPHGGGWGQPHGGGWGQPHGGGWGQPHGGGWGQGGGTHGQWNKPSKPKTSMKHVAGAAAAGAVVGGLGGYMLGSAMSRPLIHFGNDYEDRYYRENMYRYPNQVYYKPVDQYSNQNSFVHDCVNITVKQHTVTTTTKGENFTETDVKMMERVVEQMCITQYQREYQASYGRGASVIFSSPPVILLISFLIFLIVG

SEQ ID NO: 29 - protein, 250 aa, Guanaco
MVKSHMGSWILVLFVVTWSDMGLCKKRPKPGGGWNTGGSRYPGQGSPGGNRYPPQGGGGWGQPHGGGWGQPHGGGWGQPHGGGWGQPHGGGWGQGGGTHGQWNKPSKPKTSMKHVAGAAAAGAVVGGLGGYMLGSAMSRPLIHFGNDYEDRYYRENMYRYPNQVYYKPVDQYSNQNSFVHDCVNITVKQHTVTTTTKGENFTETDVKMMERVVEQMCITQYQREYQASYGRGASVIFSSPPVILLISFLI

SEQ ID NO: 30 - protein, 254 aa, vicunia
MVKSHIGSWILVLFVVTWSDMGLCKKRPKPGGGWNTGGSRYPGQGSPGGNRYPPQGGGGWGQPHGGGWGQPHGGGWGQPHGGGWGQPHGGGWGQGGGTHGQWNKPSKPKTSMKHVAGAAAAGAVVGGLGGYMLGSAMSRPLIHFGNDYEDRYYRENMYRYPNQVYYKPVDQYSNQNSFVHDCVNITVKQHTVTTTTKGENFTETDVKMMERVVEQMCITQYQREYQASYGRGASVIFSSPPVILLISFLIFLIV

SEQ ID NO: 31 - protein, 254 aa, llama
MVKSHIGSWILVLFVVTWSDVGLCKKRPKPGGGWNTGGSRYPGQGSPGGNRYPPQGGGGWGQPHGGGWGQPHGGGWGQPHGGGWGQPHGGGWGQGGGTHGQWNKPSKPKTSMKHVAGAAAAGAVVGGLGGYMLGSAMSRPLIHFGNDYEDRYYRENMYRYPNQVYYKPVDQYSNQNSFVHDCVNITVKQHTVTTTTKGENFTETDVKMMERVVEQMCITQYQREYQASYGRGASVIFSSPPVILLISFLIFLIV

SEQ ID NO: 32 - protein, 254 aa, rabbit
MAHLGYWMLLLFVATWSDVGLCKKRPKPGGGWNTGGSRYPGQSSPGGNRYPPQGGGGWGQPHGGGWGQPHGGGWGQPHGGGWGQPHGGGWGQGGGTHNQWGKPSKPKTSMKHVAGAAAAGAVVGGLGGYMLGSAMSRPLIHFGNDYEDRYYRENMYRYPNQVYYRPVDQYSNQNSFVHDCVNITVKQHTVTTTTKGENFTETDIKIMERVVEQMCITQYQQESQAAYQRAAGVLLFSSPPVILLISFLIFLIVG

SEQ ID NO: 33 - protein, 253 aa, chimp
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDQYSSQNNFVHDCVNITIKQHTVTTTTKGENFTETDVKMMERVVEQMCITQYERESQAYYQRGSSMVLFSSPPVILLISFLIFLIVG

SEQ ID NO: 34 - protein, 253 aa, Homosapien
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 35 - protein, 24 aa, Artificial
  multi-species prion peptide
  MISC_FEATURE (4)..(4): X at position 4 is P or S
  MISC_FEATURE (8)..(8): X at position 8 is G or null
  MISC_FEATURE (9)..(9): X at position 9 is G or T
RYPXQGGXXWGQPHGGGWGQPHGG

SEQ ID NO: 36 - protein, 31 aa, Artificial
  multi-species prion peptide
  MISC_FEATURE (2)..(2): X at position 2 is G or S
  MISC_FEATURE (4)..(4): X at position 4 is G or null
  MISC_FEATURE (5)..(5): X at position 5 is T, A, or S
  MISC_FEATURE (7)..(7): X at position 7 is G, N, or S
  MISC_FEATURE (10)..(10): X at position 10 is N or G
  MISC_FEATURE (13)..(13): X at position 13 is S or N
  MISC_FEATURE (18)..(18): X at position 18 is N or S
  MISC_FEATURE (19)..(19): X at position 19 is M or L
  MISC_FEATURE (22)..(22): X at position 22 is V or M
QXGXXHXQWXKPXKPKTXXKHXAGAAAAGAV

SEQ ID NO: 37 - protein, 28 aa, Artificial
  multi-species prion peptide
  MISC_FEATURE (2)..(2): X at position 2 is N or S
  MISC_FEATURE (4)..(4): X at position 4 is Y or W
  MISC_FEATURE (14)..(14): X at position 14 is Y or H
  MISC_FEATURE (18)..(18): X at position 18 is N or E
  MISC_FEATURE (23)..(23): X at position 23 is R or K
  MISC_FEATURE (25)..(25): X at position 25 is V or M
  MISC_FEATURE (26)..(26): X at position 26 is D, N or S
  MISC_FEATURE (27)..(27): X at position 27 is Q, R or E
GXDXEDRYYRENMXRYPXQVYYXPXXXY

SEQ ID NO: 38 - protein, 28 aa, Artificial
  multi-species prion peptide
  MISC_FEATURE (11)..(11): X at position 11 is M or L
  MISC_FEATURE (20)..(20): X at position 20 is L, M or I
  MISC_FEATURE (25)..(25): X at position 25 is N or S
  MISC_FEATURE (27)..(27): X at position 27 is Y or W
GAVVGGLGGYXLGSAMSRPXIHFGXDXE

SEQ ID NO: 39 - protein, 31 aa, Artificial
  multi-species prion peptide
  MISC_FEATURE (14)..(14): X at position 14 is V, M or I
  MISC_FEATURE (16)..(16): X at position 16 is M or I
  MISC_FEATURE (19)..(19): X at position 19 is R, Q, or null
  MISC_FEATURE (26)..(26): X at position 26 is I or V
  MISC_FEATURE (30)..(30): X at position 30 is Q, E, or R
  MISC_FEATURE (31)..(31): X at position 31 is R, Q, or K
TTTTKGENFTETDXKXMEXVVEQMCXTQYXX

SEQ ID NO: 40 - protein, 29 aa, Artificial
  multi-species prion peptide
  MISC_FEATURE (2)..(2): X at position 2 is R or K
  MISC_FEATURE (4)..(4): X at position 4 is V or M
  MISC_FEATURE (5)..(5): X at position 5 is D, N or S
  MISC_FEATURE (6)..(6): X at position 6 is Q, R or E
  MISC_FEATURE (8)..(8): X at position 8 is S or N
  MISC_FEATURE (9)..(9): X at position 9 is N or S
  MISC_FEATURE (12)..(12): X at position 12 is N, S, or T
  MISC_FEATURE (15)..(15): X at position 15 is H or R
  MISC_FEATURE (22)..(22): X at position 22 is V or I
  MISC_FEATURE (24)..(24): X at position 24 is Q or E
YXPXXXYXXQNXFVXDCVNITXKXHTVTT

SEQ ID NO: 41 - protein, 24 aa, Artificial
  multi-species prion peptide
RYPPPQGGGWGQPHGGGWGQPHGG

SEQ ID NO: 42 - protein, 30 aa, Artificial
  multi-species prion peptide
QGGSHSQWNKPSKPKTNLKHMAGAAAAGAV

SEQ ID NO: 43 - protein, 28 aa, Artificial
  multi-species prion peptide
GSDYEDRYYRENMYRYPEQVYYRPMDRY

SEQ ID NO: 44 - protein, 28 aa, Artificial
  multi-species prion peptide
GAVVGGLGGYMLGSAMSRPIIHFGNDWE

SEQ ID NO: 45 - protein, 31 aa, Artificial
  multi-species prion peptide
TTTTKGENFTETDIKMMERVVEQMCVTQYER

SEQ ID NO: 46 - protein, 29 aa, Artificial
  multi-species prion peptide
YRPMDEYSSQNSFVRDCVNITIKQHTVTT

SEQ ID NO: 47 - DNA, 102 bp, Artificial
  multi-species prion polynucleotide
gcgggatcccgttaccctccgcagggcggtggctggggccagccgcacggtggcggttggggccaaccgcatggcggtggtagcggcagcggtaagcttgct

SEQ ID NO: 48 - DNA, 108 bp, Artificial
  multi-species prion polynucleotide
gcgggatcccagggtggcagccatagccaatggaataaaccgtccaagccgaaaacgaacctgaaacacatggccggcgccgcggccgcgggtgcggtgaagcttgct

SEQ ID NO: 49 - DNA, 111 bp, Artificial
  multi-species prion polynucleotide
gcgggatccggtagcgattacgaagatcgttattaccgcgagaatatgtatcgttacccggagcaggtctattaccgcccgatggaccgctatggtagcggcaagcttgct

SEQ ID NO: 50 - DNA, 111 bp, Artificial
  multi-species prion polynucleotide
gcgggatccggcgccgtggtcggtggcctgggtggctatatgctgggtagcgccatgtcccgtccgattatccattttggtaatgactgggaaggtagcggcaagcttgct

SEQ ID NO: 51 - DNA, 111 bp, Artificial
  multi-species prion polynucleotide
gcgggatccaccacgaccacgaaaggcgagaacttcaccgaaacggatatcaaaatgatggaacgtgtggtcgaacagatgtgtgtgacgcagtatgagcgtaagcttgct

SEQ ID NO: 52 - DNA, 105 bp, Artificial
  multi-species prion polynucleotide
gcgggatcctatcgtccgatggatgaatactccagccagaattccttcgtgcgtgattgtgtcaacatcaccattaaacagcacaccgtcaccacgaagcttgct

SEQ ID NO: 53 - DNA, 26 bp, Artificial
  synthetic sequence
cacagcagatataagtcatcatggtg

SEQ ID NO: 54 - DNA, 37 bp, Artificial
  synthetic sequence
ggtaaagatgattaagaagatgatgaaaacaggaagg

SEQ ID NO: 55 - protein, 256 aa, white tailed deer; identical to SEQ ID NO: 8

SEQ ID NO: 56 - protein, 256 aa, white tailed deer; identical to SEQ ID NO: 9

SEQ ID NO: 57 - protein, 256 aa, red deer; identical to SEQ ID NO: 10

SEQ ID NO: 58 - protein, 264 aa, water buffalo
MVKSHIGSWILVLFVVMWSDVGLCKKRPKPGGGWNTGGSRYPGQGSPGGNRYPSQGGGGWGQPHGGGWGQPHGGGWGQPHGGGWGQPHGGGWGQPHGGGGWGQGGTHGQWNKPSKPKTNMKHMAGAAAAGAVVGGLGGYMLGSAMSRPLIHFGNDYEDRYYRENMHRYPNQVYYRPVDQYSNQNNFVHDCVNITVKEHTVTTTTKGENFTETDIKMMERVVEQMCITQYQRESQAYYQRGASVILFSSPPVILLISLLIFLIVR

SEQ ID NO: 59 - protein, 264 aa, water buffalo; identical to SEQ ID NO: 24

SEQ ID NO: 60 - protein, 255 aa, alpaca
MVKSHMGSWILVLFVVTWSDMGLCKKRPKPGGGWNTGGSRYPGQGSPGGNRYPPQGGGGWGQPHGGGWGQPHGGGWGQPHGGGWGQPHGGGWGQGGGTHGQWNKPSKPKTSMKHVAGAAAAGAVVGGLGGYMLGSAMSRPLIHFGNDYEDRYYRENMYRYPNQVYYKPVDQYSNQNSFVHDCVNITVKQHTVTTTTKGENFTETDVKMMERVVEQMCITQYQREYQASYGRGASVIFSSPPVILLISFLIFLIVG

SEQ ID NO: 61 - protein, 250 aa, guanaco; identical to SEQ ID NO: 29

SEQ ID NO: 62 - protein, 254 aa, vicunia; identical to SEQ ID NO: 30

SEQ ID NO: 63 - protein, 254 aa, llama; identical to SEQ ID NO: 31

SEQ ID NO: 64 - protein, 30 aa, Artificial
  synthetic sequence
GSRYPPQGGGWGQPHGGGWGQPHGGGSGSG

SEQ ID NO: 65 - protein, 32 aa, Artificial
  synthetic sequence
GSQGGSHSQWNKPSKPKTNLKHMAGAAAAGAV

SEQ ID NO: 66 - protein, 32 aa, Artificial
  synthetic sequence
GSGAVVGGLGGYMLGSAMSRPIIHFGNDWEGS

SEQ ID NO: 67 - protein, 32 aa, Artificial
  synthetic sequence
GSGSDYEDRYYRENMYRYPEQVYYRPMDRYGS

SEQ ID NO: 68 - protein, 31 aa, Artificial
  synthetic sequence
GSYRPMDEYSSQNSFVRDCVNITIKQHTVTT

SEQ ID NO: 69 - protein, 33 aa, Artificial
  synthetic sequence
GSTTTTKGENFTETDIKMMERVVEQMCVTQYER

SEQ ID NO: 70 - DNA, 33 bp, Artificial
  synthetic sequence
ctagaggatccgatatcaagcttactagttaat

SEQ ID NO: 71 - DNA, 102 bp, Artificial; identical to SEQ ID NO: 47
  synthetic sequence

SEQ ID NO: 72 - DNA, 102 bp, Artificial
  synthetic sequence
agcaagcttaccgctgccgctaccaccgccatgcggttggccccaaccgccaccgtgcggctggccccagccaccgccctgcggagggtaacgggatcccgc

SEQ ID NO: 73 - DNA, 108 bp, Artificial; identical to SEQ ID NO: 48
  synthetic sequence

SEQ ID NO: 74 - DNA, 108 bp, Artificial
  synthetic sequence
agcaagcttcaccgcacccgcggccgcggcgccggccatgtgtttcaggttcgttttcggcttggacggtttattccattggctatggctgccaccctgggatcccgc

SEQ ID NO: 75 - DNA, 111 bp, Artificial; identical to SEQ ID NO: 50
  synthetic sequence

SEQ ID NO: 76 - DNA, 111 bp, Artificial
  synthetic sequence
agcaagcttgccgctaccttcccagtcattaccaaaatggataatcggacgggacatggcgctacccagcatatagccacccaggccaccgaccacggcgccggatcccgc

SEQ ID NO: 77 - DNA, 111 bp, Artificial; identical to SEQ ID NO: 49
  synthetic sequence

SEQ ID NO: 78 - DNA, 111 bp, Artificial
  synthetic sequence
agcaagcttgccgctaccatagcggtccatcgggcggtaatagacctgctccgggtaacgatacatattctcgcggtaataacgatcttcgtaatcgctaccggatcccgc

SEQ ID NO: 79 - DNA, 105 bp, Artificial; identical to SEQ ID NO: 52
  synthetic sequence

SEQ ID NO: 80 - DNA, 105 bp, Artificial
  synthetic sequence
agcaagcttcgtggtgacggtgtgctgtttaatggtgatgttgacacaatcacgcacgaaggaattctggctggagtattcatccatcggacgataggatcccgc

SEQ ID NO: 81 - DNA, 111 bp, Artificial; identical to SEQ ID NO: 51
  synthetic sequence

SEQ ID NO: 82 - DNA, 111 bp, Artificial
  synthetic sequence
agcaagcttacgctcatactgcgtcacacacatctgttcgaccacacgttccatcattttgatatccgtttcggtgaagttctcgcctttcgtggtcgtggtggatcccgc

SEQ ID NO: 83 - protein, 268 aa, Artificial
  synthetic sequence
  MISC_FEATURE (2)..(2): X at position 2 is V or A
  MISC_FEATURE (5)..(5): X at position 5 is N or H
  MISC_FEATURE (6)..(6): X at position 6 is I, V, L or M
  MISC_FEATURE (8)..(8): X at position 8 is S, G, Y or C
  MISC_FEATURE (10)..(10): X at position 10 is I, L or M
  MISC_FEATURE (12)..(12): X at position 12 is V, A, or L
  MISC_FEATURE (16)..(16): X at position 16 is A, T, or V
  MISC_FEATURE (17)..(17): X at position 17 is T or M
  MISC_FEATURE (19)..(19): X at position 19 is S or T
  MISC_FEATURE (21)..(21): X at position 21 is V, I, or M
  MISC_FEATURE (23)..(23): X at position 23 is L or F
  MISC_FEATURE (27)..(27): X at position 27 is R or W
  MISC_FEATURE (33)..(33): X at position 33 is G or null
  MISC_FEATURE (39)..(39): X at position 39 is G or null
  MISC_FEATURE (47)..(47): X at position 47 is S or I
  MISC_FEATURE (55)..(55): X at position 55 is P or S
  MISC_FEATURE (59)..(59): X at position 59 is G or null
  MISC_FEATURE (60)..(60): X at position 60 is G or T
  MISC_FEATURE (76)..(76): X at position 76 is G or S
  MISC_FEATURE (84)..(84): X at position 84 is G or S
  MISC_FEATURE (92)..(92): X at position 92 is G or null
  MISC_FEATURE (93)..(93): X at position 93 is W or null
  MISC_FEATURE (94)..(94): X at position 94 is G or null
  MISC_FEATURE (95)..(95): X at position 95 is Q or null
  MISC_FEATURE (96)..(96): X at position 96 is P or null
  MISC_FEATURE (97)..(97): X at position 97 is H or null
  MISC_FEATURE (98)..(98): X at position 98 is G or null
  MISC_FEATURE (99)..(99): X at position 99 is G or null
  MISC_FEATURE (100)..(100): X at position 100 is G or null
  MISC_FEATURE (105)..(105): X at position 105 is G or S
  MISC_FEATURE (107)..(107): X at position 107 is G or or null
  MISC_FEATURE (108)..(108): X at position 108 is T, A, or S
  MISC_FEATURE (110)..(110): X at position 110 is G, N, or S
  MISC_FEATURE (113)..(113): X at position 113 is N or G
  MISC_FEATURE (116)..(116): X at position 116 is S or N
  MISC_FEATURE (121)..(121): X at position 121 is N or S
  MISC_FEATURE (122)..(122): X at position 122 is M or L
  MISC_FEATURE (125)..(125): X at position 125 is V or M
  MISC_FEATURE (142)..(142): X at position 142 is M or L
  MISC_FEATURE (151)..(151): X at position 151 is L, M or I
  MISC_FEATURE (156)..(156): X at position 156 is N or S
  MISC_FEATURE (158)..(158): X at position 158 is Y or W
  MISC_FEATURE (168)..(168): X at position 168 is Y or H
  MISC_FEATURE (172)..(172): X at position 172 is N or E
  MISC_FEATURE (177)..(177): X at position 177 is R or K
  MISC_FEATURE (179)..(179): X at position 179 is V or M
  MISC_FEATURE (180)..(180): X at position 180 is D, N or S
  MISC_FEATURE (181)..(181): X at position 181 is Q, R or E
  MISC_FEATURE (183)..(183): X at position 183 is S or N
  MISC_FEATURE (184)..(184): X at position 184 is N or S
  MISC_FEATURE (187)..(187): X at position 187 is N, S, or T
  MISC_FEATURE (190)..(190): X at position 190 is H or R
  MISC_FEATURE (197)..(197): X at position 197 is V or I
  MISC_FEATURE (199)..(199): X at position 199 is Q or E
  MISC_FEATURE (216)..(216): X at position 216 is V, M or I
  MISC_FEATURE (218)..(218): X at position 218 is M or I
  MISC_FEATURE (221)..(221): X at position 221 is R or Q
  MISC_FEATURE (228)..(228): X at position 228 is I or V
  MISC_FEATURE (232)..(232): X at position 232 is Q, E, or R
  MISC_FEATURE (233)..(233): X at position 233 is R, Q, or K
  MISC_FEATURE (235)..(235): X at position 235 is S or Y
  MISC_FEATURE (236)..(236): X at position 236 is Q or E
  MISC_FEATURE (238)..(238): X at position 238 is Y, S, F, or A
  MISC_FEATURE (239)..(239): X at position 239 is Y or Q
  MISC_FEATURE (240)..(240): X at position 240 is Q, D, or G
  MISC_FEATURE (241)..(241): X at position 241 is G or null
  MISC_FEATURE (242)..(242): X at position 242 is R or null
  MISC_FEATURE (243)..(243): X at position 243 is R or K
  MISC_FEATURE (244)..(244): X at position 244 is G, S, or A
  MISC_FEATURE (245)..(245): X at position 245 is A or S
  MISC_FEATURE (246)..(246): X at position 246 is S or G
  MISC_FEATURE (247)..(247): X at position 247 is V, T, A, M, or null
  MISC_FEATURE (248)..(248): X at position 248 is I, L, or V
  MISC_FEATURE (249)..(249): X at position 249 is L or I
  MISC_FEATURE (252)..(252): X at position 252 is S or P
  MISC_FEATURE (256)..(256): X at position 256 is I or V
  MISC_FEATURE (261)..(261): X at position 261 is F or L
  MISC_FEATURE (264)..(264): X at position 264 is F or L
  MISC_FEATURE (268)..(268): X at position 268 is G or R
MXKSXXGXWXLXLFVXXWXDXGXCKKXPKPGGXWNTGGXSRYPGQGXPGGNRYPXQGGXXWGQPHGGGWGQPHGGXWGQPHGGXWGQPHGGXXXXXXXXXGWGQXGXXHXQWXKPXKPKTXXKHXAGAAAAGAVVGGLGGYXLGSAMSRPXIHFGXDXEDRYYRENMXRYPXQVYYXPXXXYXXQNXFVXDCVNITXKXHTVTTTTKGENFTETDXKXMEXVVEQMCXTQYXXEXXAXXXXXXXXXXXXFSXPPVXLLISXLIXLIVX

(19) United States
(12) Patent Application Publication
(10) Pub. No.: US 2022/0313802 A1
(43) Pub. Date: Oct. 6, 2022

(54) COMPOSITIONS AND METHODS FOR THE TREATMENT AND PREVENTION OF PRION DISEASE

(71) Applicants: NEW YORK UNIVERSITY, New York, NY (US); INSTITUTO NACIONAL DE TECNOLOGIA AGROPECUARIA (INTA), Buenos Aires (AR); UNIVERSIDAD DE LA REPUBLICA, Montevideo (UY)

(72) Inventors: Fernando GONI, New York, NY (US); Thomas WISNIEWSKI, Staten Island, NY (US); Lucia YIM, Montevideo (UY); Jose Alejandro CHABALGOITY, Montevideo (UY); Analia ELISEI, Buenos Aires (AR)

(21) Appl. No.: 17/610,388

(22) PCT Filed: May 11, 2020
(86) PCT No.: PCT/US2020/032326

§ 371 Date: Nov. 10, 2021

Related U.S. Application Data
(60) Provisional application No. 62/846,250, filed on May 10, 2019.

(51) Int. Cl. A61K 39/00; A61P 25/28; C07K 14/47; C07K 16/28

(52) U.S. Cl. CPC A61K 39/0007; A61P 25/28; C07K 14/47; C07K 16/2872; A61K 2039/522; A61K 2039/523; A61K 2039/6037

(57) ABSTRACT

The present disclosure is directed to a multi-species prion protein or peptide thereof, and vaccine compositions comprising the same that are useful for the vaccination and treatment of mammalian subjects at risk of having or having prion disease. Also disclosed herein are isolated proteins and peptides, polymers, vaccines, animal bait or feed, antibodies, and methods of inhibiting the onset of or treating a prion disease.

This application claims the priority benefit of U.S. Provisional Patent Application Ser. No. 62/846,250, filed May 10, 2019, which is hereby incorporated by reference in its entirety.

This invention was made with government support under grant numbers NS47433 and NS073502 awarded by the National Institutes of Health. The government has certain rights in the invention.

### FIELD

The present application is directed to compositions and methods for the treatment and prevention of prion disease.

### BACKGROUND OF THE INVENTION

Prion disease is a unique category of illness, affecting both animals and humans, in which the underlying pathogenesis is related to a conformational change of a normal, self-protein called PrP to a pathological and infectious conformer. There is a need in the art for compositions and methods for the treatment and prevention of prion disease. The present application is directed to overcoming these and other deficiencies in the art.

### SUMMARY OF THE INVENTION

A first aspect of the present disclosure is directed to an isolated protein or peptide thereof comprising the amino acid sequence of:

(SEQ ID NO: 1)
MX₂X₃X₄X₅X₆GX₈WX₁₀LX₁₂LFVX₁₆X₁₇WX₁₉DX₂₁GX₂₃CKKX₂₇PKPGGX₃₃W
NTGGX₃₉SRYPGQGX₄₇PGGNRYPX₅₅QGGX₅₉X₆₀WGQPHGGGWGQPHGGX₇₆
WGQPHGGX₈₄WGQPHGGX₉₂X₉₃X₉₄X₉₅X₉₆X₉₇X₉₈X₉₉X₁₀₀GWGQX₁₀₅GX₁₀₇
X₁₀₈HX₁₁₀QWX₁₁₃KPX₁₁₆KPKTX₁₂₁X₁₂₂KHX₁₂₅AGAAAAGAVVGGLGGYX₁₄₂
LGSAMSRPX₁₅₁IHFGX₁₅₆DX₁₅₈EDRYYRENMX₁₆₈RYPX₁₇₂QVYYX₁₇₇PX₁₇₉
X₁₈₀X₁₈₁YX₁₈₃X₁₈₄QNX₁₈₇FVX₁₉₀DCVNITX₁₉₇KX₁₉₉HTVTTTTKGENFTET
DX₂₁₆KX₂₁₈ME X₂₂₁VVEQMCX₂₂₈TQYX₂₃₂X₂₃₃ EX₂₃₅X₂₃₆ AX₂₃₈X₂₃₉
X₂₄₀ X₂₄₁ X₂₄₂ X₂₄₃ X₂₄₄ X₂₄₅ X₂₄₆ X₂₄₇ X₂₄₈ X₂₄₉ FSX₂₅₂PPVX₂₅₆
LLISX₂₆₁LIX₂₆₄ LIVX₂₆₈

wherein X₂ is V or A; X₃ is K or null; X₄ is S or null; X₅ is N or H; X₆ is I, V, L or M; X₈ is S, G, Y or C; X₁₀ is I, L or M; X₁₂ is V, A, or L; X₁₆ is A, T, or V; X₁₇ is T or M; X₁₉ is S or T; X₂₁ is V, I, M; X₂₃ is L or F; X is R or W; X₃₃ is G or null; X₃₉ is G or null; X₄₇ S or I; X₅₅ is P or S; X₃₉ is G or null; X₆₀ is G or T; X₇₆ is G or S; X₈₄ is G or S; X₉₂ is G or null; X₉₃ is W or null; X₉₄ is G or null; X₉₅ is Q or null; X₉₆ is P or null; X₉₇ is H or null; X₉₈ is G or null; X₉₉ is G or null; X₁₀₀ is G or null; X₁₀₅ is G or S; X₁₀₇ is G or null; X₁₀₈ is T, A, or S; X₁₁₀ is G, N, or S; X₁₁₃ is N or G; X₁₁₆ is S or N; X₁₂₁ is N or S; X₁₂₂ is M or L; X₁₂₅ is V or M; X₁₄₂ is M or L; X₁₅₁ is L, M or I; X₁₅₆ is N or S; X₁₅₈ is Y or W; X₁₆₈ is Y or H; X₁₇₂ is N or E; X₁₇₇ is R or K; X₁₇₉ is V or M; X₁₈₀ is D, N or S; X₁₈₁ is Q, R or E; X₁₈₃ is S or N; X₁₈₄ is N or S; X₁₈₇ is N, S, or T, X₁₉₀ is H or R; X₁₉₇ is V or I; X₁₉₉ is Q or E; X₂₁₆ is V, M or I; X₂₁₈ is M or I; X₂₂₁ is R or Q; X₂₂₈ is I or V; X₂₃₂ is Q, E, or R; X₂₃₃ R, Q, or K; X₂₃₅ is S or Y; X₂₃₆ is Q or E; X₂₃₈ is Y, S, F, or A; X₂₃₉ is Y or Q; X₂₄₀ is Q, D, or G; X₂₄₁ is G or null; X₂₄₂ R or null; X₂₄₃ is R or K; X₂₄₄ is G, S, or A; X₂₄₅ is A or S; X₂₄₆ is S or G; X₂₄₇ is V, T, A, M or null; X₂₄₈ is I, L, V; X₂₄₉ is L or I; X₂₅₂ is S or P; X₂₅₆ is I or V; X₂₆₁ is F or L; X₂₆₄ is F or L; X₂₆₈ is G or R; and wherein the isolated protein or peptide thereof does not comprise an amino acid sequence of a corresponding protein of any one of SEQ ID Nos: 2-34 or a corresponding peptide thereof.

Another aspect of the present disclosure relates to a polymer of one or more peptides of SEQ ID NO: 1 as defined herein, and vaccine compositions comprising the same.

Another aspect of the present disclosure relates to an attenuated bacterium transformed with a vector expressing one or more proteins or peptides thereof of SEQ ID NO: 1 as defined herein, and vaccine compositions comprising the same.

Another aspect of the present disclosure is directed to methods of inhibiting the onset of or treat prion disease in a mammalian subject that involves administering, to the mammalian subject, a vaccine composition or delivery vehicle comprising the vaccine composition as described herein in an amount effective to inhibit the onset of or treat the prion disease in the mammalian subject.

Another aspect of the present disclosure relates to animal bait or feed comprising a vaccine composition as described herein.

Another aspect of the present disclosure relates to an isolated polynucleotide sequence encoding the protein or peptide thereof of SEQ ID NO: 1 as described herein. The present disclosure also relates to vectors containing the isolated polynucleotide sequences and host cells comprising these vectors.

Another aspect of the present disclosure relates to an isolated antibody or epitope-binding fragment thereof, where the antibody or epitope-binding fragment thereof binds to an epitope of the protein or peptide thereof comprising the amino acid sequence of SEQ ID NO: 1 as disclosed herein.

Another aspect of the present disclosure relates to an isolated antibody or epitope-binding fragment thereof, where the antibody or epitope-binding fragment thereof binds to an epitope of a polymer of a peptide fragment of SEQ ID NO: 1 as disclosed herein.

Another aspect of the present disclosure relates to a method of inhibiting the onset of or treating prion disease in a mammalian subject. This method involves administering, to the mammalian subject, the antibody or epitope-binding portion thereof as described herein in an amount effective to inhibit the onset of or treat the prion disease in the mammalian subject.

### BRIEF DESCRIPTION OF THE DRAWINGS

FIGS. 1A-1B show the amino acid sequence and numbering of SEQ ID NO:1, and comparative alignment of SEQ ID NOs:2-34. For appropriate comparison of amino acid numbering of each of SEQ ID NOs:2-34 to the total number of amino acids of SEQ ID NO:1 (268), the corresponding amino acid numbers of each of SEQ ID NOs:2-34 are shown at the end of each sequence. FIG. 1A shows amino acids 1-140 of SEQ ID NO:1 and the corresponding amino acid sequences of SEQ ID NOs:2-34. FIG. 1B shows amino acids 141-268 of SEQ ID NO:1 and the corresponding amino acid sequences of SEQ ID NOs:2-34.

FIG. 2 shows the representative cloning strategy for construction of plasmid pTECH-PrP I, also used for all PrP II-VI. MCS=multi-cloning site.

FIG. 3 shows the strategy for construction of recombinant fusion vectors expressing two tandem copies of the PrP I-VI peptides as C-terminal fusions to tetanus toxin fragment C in a live aroC attenuated vaccine strain of Salmonella LVR01, by duplicating the construct of FIG. 1; further duplications would render four tandem copies, and further eight tandem copies.

FIG. 4 shows immunoblots using monoclonal anti-PrP antibodies 7D9 and 6D11, depicting the presence of secreted multi-species PrP peptides I-VI (LVR01-PrP; x1=single copy, x2=two tandem copies) in the supernatants (SPNT ccx500) of the vaccine ready corresponding Salmonellas and the intracellular expression shown in the lysates (PPT lysated) of the same vaccine ready Salmonellas. The main bands are located at around 55-60 kDa molecular weight corresponding to the molecular weight of the TETc plus the added molecular weight of one copy of each peptide (PrPx1), or the two tandem copies of the corresponding peptides (PrPx2).

FIG. 5 shows H-E staining and HRP-anti-Salmonella immunostaining of a portion of the intestine of a mouse that was inoculated with the attenuated Salmonella vaccine containing a PrP multi-species peptide 20 hours before. Arrows show the Salmonella carrying peptides in epithelial cells facing the lumen of the intestine and included in lymphoid follicles at the bottom of the crevices.

FIGS. 6A-6B show immunoblots of prion disease samples from different species, detected by antibodies (FIG. 6A mouse IgM, IgA, IgG from plasma; and FIG. 6B mouse secretory IgA from feces) produced in mice vaccinated with the different multi-species PrP fragments. The vaccination strategy is described in Example 1. KO-PrP: knock out for PrP; Hu PrP: transgenic for Human PrP; Sh PrP: transgenic for sheep PrP; Elk PrP: transgenic for Elk-Deer Prp; All PrP: includes the wild type mouse background PrP. Hu: human Creutzfeld-Jacob Disease (CJD); De: Deer-Chronic Wasting Disease (CWD); Sh: Sheep-scrapie. The combination of PrPI, II, III, IV, V, or VI multi-species peptides (in single copy (x1) or double tandem copies (x2)) are indicated. The LVR01(−) acted as a negative background control of the naked attenuated Salmonella vaccine vector without any peptide insertion.

FIGS. 7A-7O are electron microscopy images of synthesized peptides in saline after incubation for 48 hs; after controlled polymerization with glutaraldehyde and incubated in saline for 48 hs; and after controlled glutaraldehyde hetero-polymerization with Cholera Toxin B (CTB) and incubated in saline for 48 hs; PrP I, saline (FIG. 7A); PrP I, glutaraldehyde (FIG. 7B); PrP I, glutaraldehyde and CTB (FIG. 7C); PrP II, saline (FIG. 7D); PrP II, glutaraldehyde (FIG. 7E); PrP II, glutaraldehyde and CTB (FIG. 7F); PrP III, saline (FIG. 7G); PrP III, glutaraldehyde (FIG. 7H); PrP III, glutaraldehyde and CTB (FIG. 7I); PrP IV, saline (FIG. 7J); PrP IV, glutaraldehyde (FIG. 7K); PrP IV, glutaraldehyde and CTB (FIG. 7L); PrP V, saline (FIG. 7M); PrP V, glutaraldehyde (FIG. 7N); PrP V, glutaraldehyde and CTB (FIG. 7O).

### DETAILED DESCRIPTION OF THE INVENTION

The present disclosure is directed to a multi-species prion protein or peptide thereof, and vaccine compositions comprising the same that are useful for the vaccination and treatment of mammalian subjects at risk of having or having prion disease.

A first aspect of the present disclosure is directed to an isolated protein or peptide thereof comprising the amino acid sequence of:

(SEQ ID NO: 1)
MX₂X₃X₄X₅X₆GX₈WX₁₀LX₁₂LFVX₁₆X₁₇WX₁₉DX₂₁GX₂₃CKKX₂₇PKPGGX₃₃W
NTGGX₃₉SRYPGQGX₄₇PGGNRYPX₅₅QGGX₅₉X₆₀WGQPHGGGWGQPHGGX₇₆
WGQPHGGX₈₄WGQPHGGX₉₂X₉₃X₉₄X₉₅X₉₆X₉₇X₉₈X₉₉X₁₀₀GWGQX₁₀₅GX₁₀₇
X₁₀₈HX₁₁₀QWX₁₁₃KPX₁₁₆KPKTX₁₂₁X₁₂₂KHX₁₂₅AGAAAAGAVVGGLGGYX₁₄₂
LGSAMSRPX₁₅₁IHFGX₁₅₆DX₁₅₈EDRYYRENMX₁₆₈RYPX₁₇₂QVYYX₁₇₇PX₁₇₉
X₁₈₀X₁₈₁YX₁₈₃X₁₈₄QNX₁₈₇FVX₁₉₀DCVNITX₁₉₇KX₁₉₉HTVTTTTKGENFTET
DX₂₁₆KX₂₁₈ME X₂₂₁VVEQMCX₂₂₈TQYX₂₃₂X₂₃₃ EX₂₃₅X₂₃₆ AX₂₃₈X₂₃₉
X₂₄₀ X₂₄₁ X₂₄₂ X₂₄₃ X₂₄₄ X₂₄₅ X₂₄₆ X₂₄₇ X₂₄₈ X₂₄₉ FSX₂₅₂PPVX₂₅₆
LLISX₂₆₁LIX₂₆₄ LIVX₂₆₈

wherein X₂ is V or A; X₃ is K or null; X₄ is S or null; X₅ is N or H; X₆ is I, V, L or M; X₈ is S, G, Y or C; X₁₀ is I, L or M; X₁₂ is V, A, or L; X₁₆ is A, T, or V; X₁₇ is T or M; X₁₉ is S or T; X₂₁ is V, I, M; X₂₃ is L or F; X₂₇ is R or W; X₃₃ is G or null; X₃₉ is G or null; X₄₇ S or I; X₅₅ is P or S; X₅₉ is G or null; X₆₀ is G or T; X₇₆ is G or S; X₈₄ is G or S; X₉₂ is G or null; X₉₃ is W or null; X₉₄ is G or null; X₉₅ is Q or null; X₉₆ is P or null; X₉₇ is H or null; X₉₈ is G or null; X₉₉ is G or null; X₁₀₀ is G or null; X₁₀₅ is G or S; X₁₀₇ is G or null; X₁₀₈ is T, A, or S; X₁₁₀ is G, N, or S; X₁₁₃ is N or G; X₁₁₆ is S or N; X₁₂₁ is N or S; X₁₂₂ is M or L; X₁₂₅ is V or M; X₁₄₂ is M or L; X₁₅₁ is L, M or I; X₁₅₆ is N or S; X₁₅₈ is Y or W; X₁₆₈ is Y or H; X₁₇₂ is N or E; X₁₇₇ is R or K; X₁₇₉ is V or M; X₁₈₀ is D, N or S; X₁₈₁ is Q, R or E; X₁₈₃ is S or N; X₁₅₄ is N or S; X₁₈₇ is N, S, or T, X₁₉₀ is H or R; X₁₉₇ is V or I; X₁₉₉ is Q or E; X₂₁₆ is V, M or I; X₂₁₈ is M or I; X₂₂₁ is R or Q; X₂₂₈ is I or V; X₂₃₂ is Q, E, or R; X₂₃₃ R, Q, or K; X₂₃₅ is S or Y; X₂₃₆ is Q or E; X₂₃₈ is Y, S, F, or A; X₂₃₉ is Y or Q; X₂₄₀ is Q, D, or G; X₂₄₁ is G or null; X₂₄₂ R or null; X₂₄₃ is R or K; X₂₄₄ is G, S, or A; X₂₄₅ is A or S; X₂₄₆ is S or G; X₂₄₇ is V, T, A, M or null; X₂₄₈ is I, L, V; X₂₄₉ is L or I; X₂₅₂ is S or P; X₂₅₆ is I or V; X₂₆₁ is F or L; X₂₆₄ is F or L; X₂₆₈ is G or R. The isolated protein or peptide thereof does not comprise an amino acid sequence of a corresponding protein of any one of SEQ ID Nos: 2-34 or a corresponding peptide thereof.

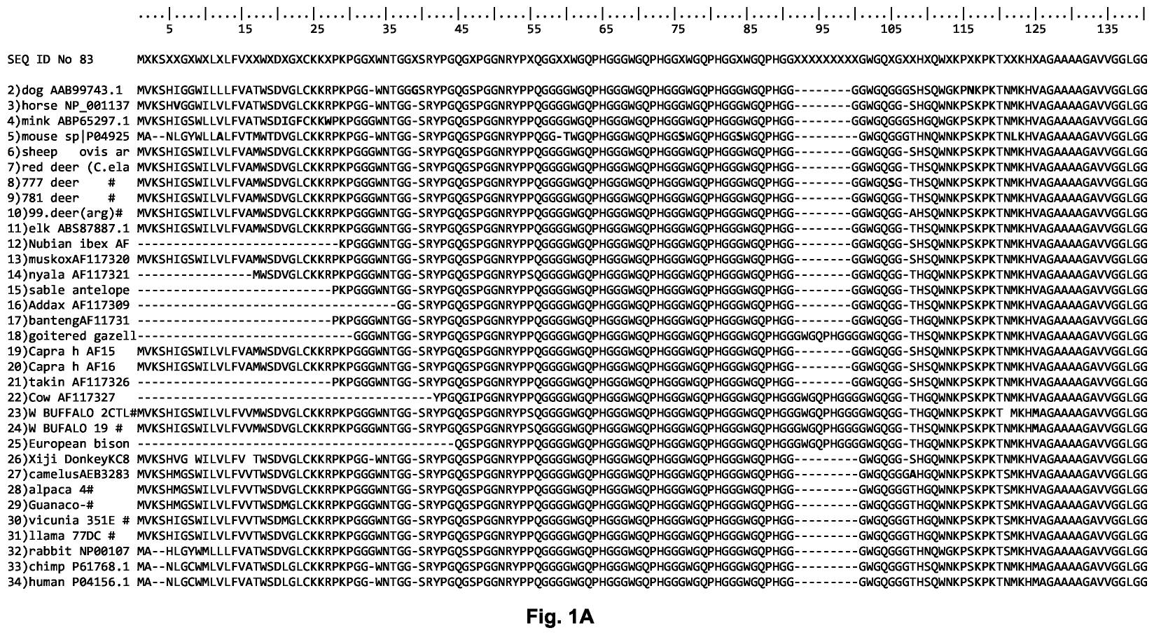

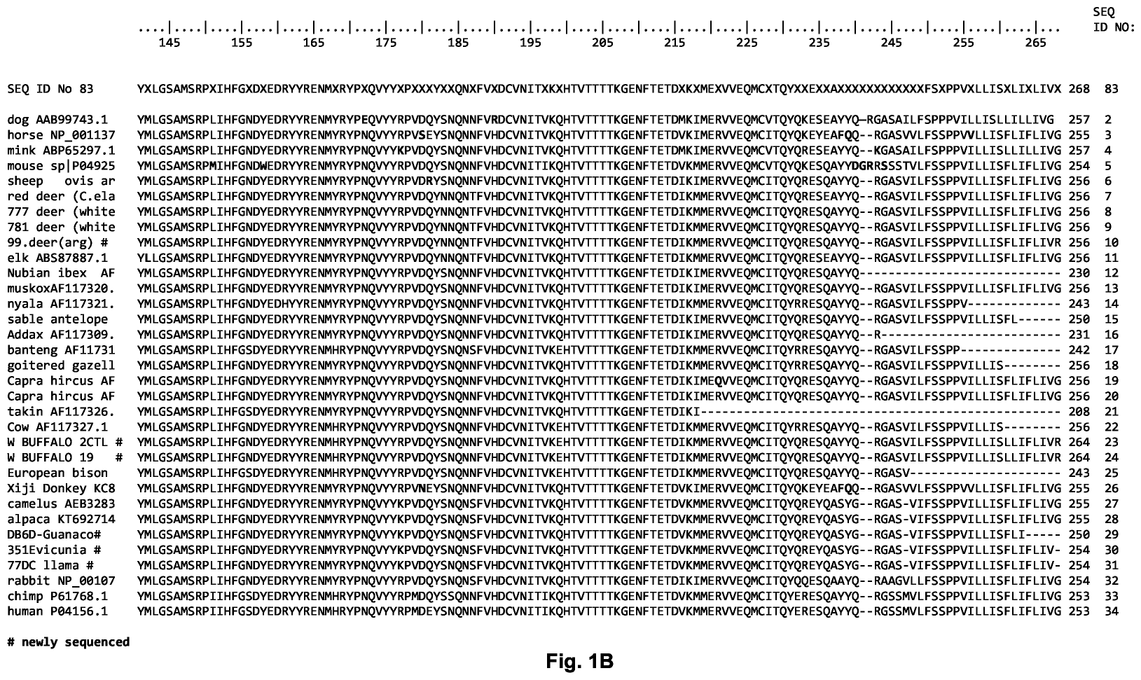

The protein sequence of SEQ ID NO: 1 is derived from an alignment of prion protein sequences from 33 different species as shown in FIG. 1. Thus, the amino acid sequence of SEQ ID NO: 1 represents the fusion of all conserved sequences of prion proteins required for structure across species. The residues of variability identified throughout the amino acid sequence of SEQ ID NO: 1 are individually defined as being an amino acid residue that is found in a naturally occurring sequence of one or more different species.

In accordance with this aspect of the present disclosure the isolated protein or peptide thereof does not have the same sequence as any corresponding naturally occurring prion protein or peptide derived therefrom. A “corresponding protein” or “corresponding peptide thereof” refers to a protein or peptide thereof that is of the same length as the protein or peptide thereof of the present disclosure. Upon alignment of the isolated protein or peptide thereof of SEQ ID NO: 1 to any corresponding naturally occurring prion protein or peptide thereof (e.g., any one of SEQ ID Nos: 2-34, as per FIG. 1), the isolated protein or peptide thereof of the disclosure comprises at least one single amino acid variation as compared to its corresponding naturally occurring prion protein sequence or peptide sequence thereof. In another embodiment, the isolated protein or peptide thereof comprises at least two single amino acid variations from any naturally occurring corresponding prion protein sequence or peptide sequence derived therefrom when the sequences are aligned as per FIG. 1. In another embodiment, the isolated protein or peptide thereof comprises at least three single amino acid variations from any naturally occurring corresponding prion protein sequence or peptide sequence derived therefrom when the sequences are aligned. In one embodiment, the isolated protein or peptide thereof comprises at least 4, at least 5, at least 6, at least 7, at least 8, at least 9, or at least 10 single amino acid variations from any naturally occurring prion protein sequence or peptide sequence derived therefrom. As referred to herein, an “amino acid variation” encompasses an amino acid substitution, deletion, or insertion.

In one embodiment, the isolated protein or peptide thereof, comprises the amino acid sequence of SEQ ID NO: 1 or a peptide thereof, wherein each of the one or more single amino acid variations, relative to any corresponding naturally occurring prion protein or peptide thereof, are independently selected from residues occurring in different species. For example, the isolated protein or peptide thereof may comprise sequence identity to a naturally occurring prion protein sequence at all but three amino acid residues. The variant residue at each of these three amino acid residue positions is selected from a different species, such that the resulting amino acid sequence is a compilation of at least three different prion sequences derived from three different species, rendering it a multi-species prion protein or peptide thereof.

In one embodiment, the isolated peptide as disclosed herein is at least 10 amino acid residues in length. In another embodiment, the isolated peptide as disclosed herein is at least 15 amino acid residues in length. In another embodiment, the isolated peptide disclosed herein is at least 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length.

In one embodiment, the isolate peptide as disclosed herein is no more than 50 amino acid residues in length. In another embodiment, the isolated peptide as disclosed herein is no more than 55 amino acid residues in length. In another embodiment, the isolated peptide as disclosed herein is no more than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 amino acid residues in length.

An exemplary isolated peptide of the present disclosure comprises the amino acid sequence of amino acid residues 52-75 of SEQ ID NO: 1. This peptide has the amino acid sequence of RYPX₅₅QGGX₅₉X₆₀WGQPHGGGWGQPHGG (SEQ ID NO: 35). For avoidance of confusion, the residues of variability in this and all exemplary peptides disclosed herein are identified by their position in the full-length protein of SEQ ID NO: 1. Thus, as described supra, the amino acid residue at position X₅₅ in the aforementioned exemplary peptide of SEQ ID NO: 35 is selected from P or S; X₅₉ is selected from G or null; and X₆₀ is selected from G or T. In one embodiment, the isolated peptide of the present disclosure comprises the amino acid sequence of RYPPPQGGGWGQPHGGGWGQPHGG (PrP I; SEQ ID NO: 41).

Another exemplary isolated peptide of the present disclosure comprises the amino acid sequence of amino acid residues 104-134 of SEQ ID NO: 1. This peptide has the amino acid sequence of QX₁₀₅GX₁₀₇X₁₀₈HX₁₁₀QWX₁₁₃ KPX₁₁₆KPKTX₁₂₁X₁₂₂KHX₁₂₅AGAAAAGAV (SEQ ID NO: 36), where X₁₀₅ is selected from G or S; X₁₀₇ is selected from G or null; X₁₀₈ is selected from T, A, or S; X₁₁₀ is selected from G, N, or S; X₁₁₃ is selected from N or G; X₁₁₆ is selected from S or N; X₁₂₁ is selected from N or S; X₁₂₂ is selected from M or L; and X₁₂₅ is selected from V or M. In one embodiment, the isolated peptide of the present disclosure comprises the amino acid sequence of

(PrPII; SEQ ID NO: 42)
QGGSHSQWNKPSKPKTNLKHMAGAAAAGAV.

Another exemplary isolated peptide of the present disclosure comprises the amino acid sequence of amino acid residues 155-182 of SEQ ID NO: 1. This peptide has the amino acid sequence of GX₁₅₆DX₁₅₈EDRYYRENMX₁₆₈RYPX₁₇₂QVYYX₁₇₇PX₁₇₉X₁₈₀X₁₈₁Y (SEQ ID NO: 37), where X₁₅₆ is selected from N or S; X₁₅₈ is selected from Y or W; X₁₆₈ is selected from Y or H; X₁₇₂ is selected from N or E; X₁₇₇ is selected from R or K; X₁₇₉ is selected from V or M; X₁₈₀ is selected from D, N or S; and X₁₈₁ is selected from Q, R or E. In one embodiment, the isolated peptide of the present disclosure comprises the amino acid sequence of GSDYEDRYYRENMYRYPEQVYYRPMDRY (PrP IV; SEQ ID NO: 43).

Another exemplary isolated peptide of the present disclosure comprises the amino acid sequence of amino acid residues 132-159 of SEQ ID NO: 1. This peptide has the amino acid sequence of GAVVGGLGGYX₁₄₂L GSAMSRP X₁₅₁IHFGX₁₅₆DX₁₅₈E (SEQ ID NO: 38), where X₁₄₂ is selected from M or L; X₁₅₁ is selected from L, M or I; X₁₅₆ is selected from N or S; X₁₅₈ is selected from Y or W. In one embodiment, the isolated peptide of the present disclosure comprises the amino acid sequence of

(PrP III; SEQ ID NO: 44)
GAVVGGLGGYMLGSANISRPIIHFGNDWE.

Another exemplary isolated peptide of the present disclosure comprises the amino acid sequence of amino acid residues 203-233 of SEQ ID NO: 1. This peptide has the amino acid sequence of TTTTKGENFTETD X₂₁₆KX₂₁₈ME X₂₂₁VVEQMCX₂₂₈TQYX₂₃₂X₂₃₃ (SEQ ID NO: 39), where X₂₁₆ is selected from V, M or I; X₂₁₈ is selected from M or I; X₂₂₁ is selected from R, Q, or null; X₂₂₈ is selected from I or V; X₂₃₂ is selected from Q, E, or R; and X₂₃₃ is selected from R, Q, or K. In one embodiment, the isolated peptide of the present disclosure comprises the amino acid sequence of

(PrP: VI; SEQ ID NO: 45)
TTTTKGENFTETDIKMMERVVEQMCVTQYER.

Another exemplary isolated peptide of the present disclosure comprises the amino acid sequence of amino acid residues 176-204 of SEQ ID NO: 1. This peptide has the amino acid sequence of YX₁₇₇PX₁₇₉X₁₈₀X₁₈₁YX₁₈₃X₁₈₄QNX₁₈₇ FVX₁₉₀DCVNITX₁₉₇KX₁₉₉HTVTT (SEQ ID NO: 40), where X₁₇₇ is selected from R or K; X₁₇₉ is selected from V or M; X₁₈₀ is selected from D, N or S; X₁₈₁ is selected from Q, R or E; X₁₈₃ is selected from S or N; X₁₈₄ is selected from N or S; X₁₈₇ is selected from N, S, or T, X₁₉₀ is selected from H or R; X₁₉₇ is selected from V or I; and X₁₉₉ is selected from Q or E. In one embodiment, the isolated peptide of the present disclosure comprises the amino acid sequence of YRPMDEYSSQNSFVRDCVNITIKQHTVTT (PrPV; SEQ ID NO:46).

The amino acid residues of the isolated protein or peptides there as described herein may be in either L- or D-form. In one embodiment, the isolated protein or peptide thereof as described herein is comprised of D-form amino acid residues. In another embodiment, the isolated protein or peptide thereof as described herein is comprised of D-form amino acid residues. D-form peptides have a higher stability than L-form peptides in vivo. In another embodiment, the isolated protein or peptide thereof is N- or C-terminally coupled to a polylysine or polyaspartate segment. In another embodiment, the C-terminal residue of the isolated protein or peptide thereof is amidated.

The isolated protein or peptide thereof as described herein may further comprise an adapter sequence on its amino terminus, carboxy terminus, or both termini to ensure flexibility and proper conformation of the epitopes of the protein or peptide independently of their proximity to the amino or carboxy termini. Suitable adapter sequences for this purpose include, without limitation, short glycine-serine adapter sequences, such as -GSG or -GSGSG. In another embodiment, when the isolated protein or peptide thereof is polymerized as described herein, the isolated protein or peptide thereof should have a K (lysine) residue after the glycine at the amino end (GKSG) and/or before the glycine at the carboxyl end (GSKG) to maximize the polymerization with glutaraldehyde avoiding the amino-end Schiff base formation on a monomer versus the covalent link to another peptide.

Another aspect of the present disclosure is directed to polynucleotides encoding the isolated protein and peptides thereof as described herein. In one embodiment, the polynucleotide encodes the amino acid sequence of SEQ ID NO: 1 or a peptide thereof. In another embodiment, the polynucleotide encodes the amino acid sequence of SEQ ID NO: 35. In another embodiment, the polynucleotide encodes the amino acid sequence of SEQ ID NO: 36. In another embodiment, the polynucleotide encodes the amino acid sequence of SEQ ID NO: 37. In another embodiment, the polynucleotide encodes the amino acid sequence of SEQ ID NO: 38. In another embodiment, the polynucleotide encodes the amino acid sequence of SEQ ID NO: 39. In another embodiment, the polynucleotide encodes the amino acid sequence of SEQ ID NO: 40.

In another embodiment, the polynucleotide encodes the amino acid sequence of SEQ ID NO: 41 (PrP I). An exemplary polynucleotide encoding the amino acid sequence of SEQ ID NO: 41 has the nucleotide sequence of SEQ ID NO: 47.

(SEQ ID NO: 47)
5′-
GCGGGATCCCGTTACCCTCCGCAGGGCGGTGGCTGGGGCCAGCCGCACG
GTGGCGGTTGGGGCCAACCGCATGGCGGTGGTAGCGGCAGCGGTAAGCT
TGCT-3′

In another embodiment, the polynucleotide encodes the amino acid sequence of SEQ ID NO: 42 (PrP II). An exemplary polynucleotide encoding the amino acid sequence of SEQ ID NO: 42 has the nucleotide sequence of SEQ ID NO: 48.

(SEQ ID NO: 48)
5′-
GCGGGATCCCAGGGTGGCAGCCATAGCCAATGGAATAAACCGTCCAAGCC
GAAAACGAACCTGAAACACATGGCCGGCGCCGCGGCCGCGGGTGCGGTGA
AGCTTGCT-3′

In another embodiment, the polynucleotide encodes the amino acid sequence of SEQ ID NO: 43 (PrP IV). An exemplary polynucleotide encoding the amino acid sequence of SEQ ID NO: 43 has the nucleotide sequence of SEQ ID NO: 49.

(SEQ ID NO: 49)
5′-
GCGGGATCCGGTAGCGATTACGAAGATCGTTATTACCGCGAGAATATGTA
TCGTTACCCGGAGCAGGTCTATTACCGCCCGATGGACCGCTATGGTAGCG
GCAAGCTTGCT-3′

In another embodiment, the polynucleotide encodes the amino acid sequence of SEQ ID NO: 44 (PrP III). An exemplary polynucleotide encoding the amino acid sequence of SEQ ID NO: 44 has the nucleotide sequence of SEQ ID NO: 50.

(SEQ ID NO: 50)
5′-
GCGGGATCCGGCGCCGTGGTCGGTGGCCTGGGTGGCTATATGCTGGGTAG
CGCCATGTCCCGTCCGATTATCCATTTTGGTAATGACTGGGAAGGTAGCG
GCAAGCTTGCT-3′

In another embodiment, the polynucleotide encodes the amino acid sequence of SEQ ID NO: 45 (PrP VI). An exemplary polynucleotide encoding the amino acid sequence of SEQ ID NO: 45 has the nucleotide sequence of SEQ ID NO: 51.

(SEQ ID NO: 51)
5′-
GCGGGATCCACCACGACCACGAAAGGCGAGAACTTCACCGAAACGGATAT
CAAAATGATGGAACGTGTGGTCGAACAGATGTGTGTGACGCAGTATGAGC
GTAAGCTTGCT-3′

In another embodiment, the polynucleotide encodes the amino acid sequence of SEQ ID NO: 46 (PrP V). An exemplary polynucleotide encoding the amino acid sequence of SEQ ID NO: 46 has the nucleotide sequence of SEQ ID NO: 52.

(SEQ ID NO: 52)
5′-
GCGGGATCCTATCGTCCGATGGATGAATACTCCAGCCAGAATTCCTTCGT
GCGTGATTGTGTCAACATCACCATTAAACAGCACACCGTCACCACGAAGC
TTGCT-3′

The isolated protein or peptide thereof as described supra may be linked in-frame to an adjuvant polypeptide. The adjuvant polypeptide can be any adjuvant polypeptide known in the art, including, but not limited to, cholera toxin B, flagellin, human papillomavirus L1 or L2 protein, herpes simplex glycoprotein D (gD), complement C4 binding protein, TL4 ligand, influenza HA, Gb3, and IL-1β. The isolated protein or peptide thereof as described herein may be linked directly to the adjuvant polypeptide or coupled to the adjuvant by way of a short linker sequence. Suitable linker sequences include glycine-rich (e.g. G₃₋₅) or serine-rich (e.g., GSG, GSGS, GSGSG, GS_{N}G) linker sequences or other flexible immunoglobulin linkers as disclosed in U.S. Pat. No. 5,516,637 to Huang et al, which is hereby incorporated by reference in its entirety.

In another embodiment, the isolated protein or peptide thereof as described supra may be conjugated to an immunogenic carrier molecule. The immunogenic carrier molecule can be covalently or non-covalently bonded to the protein or peptide thereof. Suitable immunogenic carrier molecules include, but are not limited to, serum albumins, chicken egg ovalbumin, keyhole limpet hemocyanin, tetanus toxoid, thyroglobulin, pneumococcal capsular polysaccharides, CRM 197, immunoglobulin molecules, alum, and meningococcal outer membrane proteins. Other suitable immunogenic carrier molecules include T-cell epitopes, such as tetanus toxoid (e.g., the P2 and P30 epitopes), Hepatitis B surface antigen, pertussis, toxoid, diphtheria toxoid, measles virus F protein, Chlamydia trachomatis major outer membrane protein, Plasmodium falciparum: circumsporozite T, P. falciparum CS antigen, Schistosoma mansoni triose phosphate isomersae, Escherichia coli TraT, and Influenza virus hemagluttinin (HA). Other suitable immunogenic carrier molecules include promiscuous T helper cell epitopes which are derived from hepatitis B virus, Bordetella pertussis, Clostridium tetani, Pertusaria trachythallina, E. coli, Chlamydia trachomatis, Diphtheria, P. falciparum, and Schistosoma mansoni (see U.S. Pat. No. 6,906,169 to Wang; U.S. Patent Application Publication No. 20030068325 to Wang, and WO/2002/096350 to Wang, which are hereby incorporated by reference in their entirety). Yet other suitable carriers include T-helper cell epitopes derived from tetanus toxin, cholera toxin B, pertussis toxin, diphtheria toxin, measles virus F protein, hepatitis B virus surface antigen, C. trachomitis major outer membrane protein, P. falciparum circumsporozoite, S. mansoni triose phosphate isomerase, or E. coli TraT (see WO01/42306 to Chain, which is hereby incorporated by reference in its entirety).

The isolated protein and peptide thereof of the present disclosure can be linked to immunogenic carrier molecules by chemical crosslinking. Techniques for linking a peptide immunogen to an immunogenic carrier molecule include the formation of disulfide linkages using N-succinimidyl-3-(2-pyridyl-thio) propionate (SPDP) and succinimidyl 4-(N-maleimidomethyl)cyclohexane-1-carboxylate (SMCC) (if the peptide lacks a sulfhydryl group, this can be provided by addition of a cysteine residue). These reagents create a disulfide linkage between themselves and peptide cysteine residues on one protein, and an amide linkage through the epsilon-amino on a lysine, or other free amino group in other amino acids. A variety of such disulfide/amide-forming agents are described by Jansen et al., “Immunotoxins: Hybrid Molecules Combining High Specificity and Potent Cytotoxicity,” Immun Rev 62:185-216 (1982), which is hereby incorporated by reference in its entirety. Other bifunctional coupling agents form a thioether rather than a disulfide linkage. Many of these thio-ether-forming agents are commercially available and include reactive esters of 6-maleimidocaproic acid, 2-bromoacetic acid, 2-iodoacetic acid, and 4-(N-maleimido-methyl)cyclohexane-1-carboxylic acid. The carboxyl groups can be activated by combining them with succinimide or 1-hydroxyl-2-nitro-4-sulfonic acid, sodium salt.

Another aspect of the present disclosure relates to a polymer of the isolated protein or peptide thereof of SEQ ID NO: 1 as disclosed herein. Polymerization of the isolated protein or peptide thereof alone or conjugated to an adjuvant polypeptide or immunogenic carrier molecule can be achieved using standard techniques known in the art. In one embodiment, the protein or peptide thereof is polymerized by a reaction with a cross linking reagent. Suitable cross-linking reagents include, but are not limited to glutaraldehyde and 1-Ethyl-3-[3-dimethylaminopropyl]carbodiimide hydrochloride (EDC). Alternatively, the isolated protein or peptide thereof can be polymerized by cysteine oxidation induced disulfide cross linking.

In one embodiment, a polymer as disclosed herein is a homopolymer, which is made of repeating units (i.e., monomers) of a single isolated protein or peptide thereof of SEQ ID NO: 1 as described herein. A monomer of a polymer as described herein is a single isolated protein or peptide thereof of SEQ ID NO: 1. In another embodiment, a polymer as disclosed herein is a heteropolymer, which is made of repeating monomers of two or more different isolated proteins or peptides thereof of SEQ ID NO: 1.

In one embodiment, the polymer as disclosed herein is made of repeating units of the peptide having the amino acid sequence of SEQ ID NO: 35. In another embodiment, the polymer as disclosed herein is made of repeating units of the peptide having the amino acid sequence of SEQ ID NO: 36. In another embodiment, the polymer as disclosed herein is made of repeating units of the peptide having the amino acid sequence of SEQ ID NO: 37. In another embodiment, the polymer as disclosed herein is made of repeating units of the peptide having the amino acid sequence of SEQ ID NO: 38. In another embodiment, the polymer as disclosed herein is made of repeating units of the peptide having the amino acid sequence of SEQ ID NO: 39. In another embodiment, the polymer as disclosed herein is made of repeating units of the peptide having the amino acid sequence of SEQ ID NO: 40. In another embodiment, the polymer as disclosed herein is comprised of repeating units of two or more peptides selected from the peptides of SEQ ID Nos: 35-40.

In one embodiment, the polymer as described herein is made via a controlled glutaraldehyde crosslinking reaction. Polymers made in this manner, including homopolymers and heteropolymers comprise, monomers (e.g., peptides as described herein) that are each covalently linked to another monomer of the polymer by a glutaraldehyde molecule as described in Goni et al., PLoS One. 5(10):e13391 (2010) and Goni et al., Sci Rep. 7(1):9881 (2017), which are hereby incorporated by reference in their entirety.

Another aspect of the present disclosure relates to a vaccine composition comprising one or more polymers as described herein and a pharmaceutically acceptable carrier. The polymers of the vaccine composition may be homopolymers, heteropolymers, or a mixture thereof. In one embodiment, the vaccine composition comprises a single polymer. In another embodiment, the vaccine composition comprises two or more different polymers. In another embodiment, the vaccine composition comprises three of more different polymers. In another embodiment, the vaccine composition comprises up to six different polymers.

In one embodiment, the vaccine composition comprises a polymer of the peptide having the amino acid sequence of SEQ ID NO: 35, alone or in combination with a different polymer. In another embodiment, the vaccine composition comprises a polymer of the peptide having the amino acid sequence of SEQ ID NO: 36, alone or in combination with a different polymer. In another embodiment, the vaccine composition comprises a polymer of the peptide having the amino acid sequence of SEQ ID NO: 37, alone or in combination with a different polymer. In another embodiment, the vaccine composition comprises a polymer of the peptide having the amino acid sequence of SEQ ID NO: 38, alone or in combination with a different polymer. In another embodiment, the vaccine composition comprises a polymer of the peptide having the amino acid sequence of SEQ ID NO: 39, alone or in combination with a different polymer. In another embodiment, the vaccine composition comprises a polymer of the peptide having the amino acid sequence of SEQ ID NO: 40, alone or in combination with a different polymer. In another embodiment, the vaccine composition as disclosed herein comprises one or more heteropolymers, where each heteropolymer comprises two or more peptides selected from the peptides of SEQ ID Nos: 35-40.

The vaccine composition comprising one or more polymers described herein may further contain, in addition to the polymers, other pharmaceutically acceptable components (see REMINGTON'S PHARMACEUTICAL SCIENCE (19th ed., 1995), which is hereby incorporated by reference in its entirety). The incorporation of such pharmaceutically acceptable components depends on the intended mode of administration and therapeutic application of the pharmaceutical composition. Typically, however, the vaccine composition will include a pharmaceutically-acceptable, non-toxic carrier or diluent, which are defined as vehicles commonly used to formulate pharmaceutical compositions for animal or human administration. The diluent is selected so as not to affect the biological activity of the composition. Exemplary carriers or diluents include distilled water, physiological phosphate-buffered saline, Ringer's solutions, dextrose solution, ammonium bicarbonate, and Hank's solution.

Vaccine compositions can also include large, slowly metabolized macromolecules such as proteins, polysaccharides such as chitosan, polylactic acids, polyglycolic acids and copolymers (such as latex functionalized sepharose, agarose, cellulose), polymeric amino acids, amino acid copolymers, and lipid aggregates (such as oil droplets or liposomes).

The vaccine composition of the present invention can further contain an adjuvant. An “adjuvant” as used herein is a substance that augments, stimulates, activates, or potentiates an immune response against a prion protein at either the cellular or humoral level. The adjuvant may be conjugated or cross-linked to the immunogen. Alternatively, the adjuvant is not conjugated to the prion protein of SEQ ID NO: 1, peptide thereof, or polymer thereof, but is added as an exogenous adjuvant/emulsion formulation which maximizes the immune response to the protein, peptide thereof, or polymer thereof. Suitable adjuvants include, without limitation, aluminum salt, cholera toxin subunit B, heat labile enterotoxin, flagellin, Freund's complete adjuvant, Freund's incomplete adjuvant, lysolecithin, pluronic polyols, polyanions, an oil-water emulsion, dinitrophenol, iscomatrix, influenza HA, Gb3, and liposome polycation DNA particles. In one embodiment, the adjuvant is one that maximizes the mucosal immune response. Preferred adjuvants are those which are shown to promote mucosal immunity with minimal or at least acceptable side effects.

One class of preferred adjuvants is aluminum salts, such as aluminum hydroxide, aluminum phosphate, or aluminum sulfate. Such adjuvants can be used with or without other specific immunostimulating agents such as MPL or 3-DMP, QS-21, flagellin, polymeric or monomeric amino acids such as polyglutamic acid or polylysine, or pluronic polyols. Oil-in-water emulsion formulations are also suitable adjuvants that can be used with or without other specific immunostimulating agents such as muramyl peptides (e.g., N-acetylmuramyl-L-threonyl-D-isoglutamine (thr-MDP), N-acetyl-normuramyl-L-alanyl-D-isoglutamine (nor-MDP), N-acetylmuramyl-L-alanyl-D-isoglutaminyl-L-alanine-2-(1′-2′dipalmitoyl-sn- -glycero-3-hydroxyphosphoryloxy)-ethylamine (MTP-PE), N-acetylglucsaminyl-N-acetylmuramyl-L-Al-D-isoglu-L-Ala-dipalmitoxy propylamide (DTP-DPP) Theramide™, or other bacterial cell wall components). A suitable oil-in-water emulsion is MF59 (containing 5% Squalene, 0.5% Tween 80, and 0.5% Span 85 (optionally containing various amounts of MTP-PE) formulated into submicron particles using a microfluidizer such as Model 110Y microfluidizer (Microfluidics, Newton Mass.) as described in WO90/14837 to Van Nest et al., which is hereby incorporated by reference in its entirety. Other suitable oil-in-water emulsions include SAF (containing 10% Squalene, 0.4% Tween 80, 5% pluronic-blocked polymer L121, and thr-MDP, either microfluidized into a submicron emulsion or vortexed to generate a larger particle size emulsion) and Ribi™ adjuvant system (RAS; containing 2% squalene, 0.2% Tween 80, and one or more bacterial cell wall components). Another class of preferred adjuvants is saponin adjuvants, such as Stimulon™ (QS-21) or particles generated therefrom such as ISCOMs (immunostimulating complexes) and ISCOMATRIX. Other suitable adjuvants include incomplete or complete Freund's Adjuvant (IFA), cytokines, such as interleukins (IL-1, IL-2, and IL-12), macrophage colony stimulating factor (M-CSF), lysolecithin, tumor necrosis factor (TNF), and liposome polycation DNA particles. Such adjuvants are generally available from commercial sources.

In another embodiment, the vaccine composition is packaged into a delivery vehicle. Suitable delivery vehicles include, but are not limited to biodegradable microspheres, microparticles, nanoparticles, liposomes, collagen minipellets, micelles, and cochleates. Cochleates are stable phospholipid calcium precipitates distinct from liposomes, which provide protection for vaccines from harsh acid and degradative environments thereby allowing efficient delivery by mucosal routes (Mannino and Gould-Fogerite, Pharm. Biotechnol., 6:363-87 (1995), which is hereby incorporated by reference in its entirety).

Another aspect of the present disclosure is directed to an attenuated microorganism transformed with a vector expressing one or more proteins or peptides thereof of SEQ ID NO: 1 as disclosed herein.

An “attenuated” microorganism is an organism with reduced virulence (infectivity). Because of their reduced virulence, attenuated microorganisms are suitable for use as antigen delivery vectors (sometimes with adjuvant properties) in vaccines. Methods for attenuating microorganisms are well known in the art. See, e.g., Chabalgoity et al., Vaccine, 19:460-469 (20000; Pasetti et al. Clin Immunol 92:76-89 (1999) and Hoiseth et al. Nature, 291:238-239 (1981), which are hereby incorporated by reference in their entirety. In one embodiment, the attenuated microorganism is an attenuated bacterium. Suitable attenuated bacterium include, without limitation, an attenuated strain of Salmonella, e.g., an attenuated strain of S. typhimurium, such as LVR01 (Chabalgoity et al., Vaccine, 19:460-469 (2000), which is hereby incorporated by reference in its entirety), LVR03 (a mouse-adapted derivative of LVR01), or SL3261 (Hoiseth et al., Nature, 291:238-239 (1981), Salmonella typhi CVD908-htrA (Tacket et al., Infect Immun., 68(3): 1196-1201 (2000), or Salmonella typhi Ty21a (Pasetti et al., Clin Immunol 92:76-89 (1999), which is hereby incorporated by reference in its entirety). Another suitable attenuated bacterium for use in accordance with the present disclosure is an attenuated strain of Shigella (see e.g., Chatfield et al., Vaccine, 10:53-60 (1992), which is hereby incorporated by reference in its entirety), such as WRSS1 (see e.g., Kotloff et al., Infect. Immun., 70:2016-21 (2002), which is hereby incorporated by reference in its entirety).

In one embodiment, the attenuated bacterium comprises a vector expressing the one or more proteins or peptides of interest, where each protein or peptide is expressed as single units from the vector. In other words, the vector comprises a single nucleotide sequence expressing a single protein or peptide of interest. In another embodiment, the attenuated bacterium comprises a vector expressing tandemly repeated units of the protein or peptide of interest. In this embodiment, the vector comprises a nucleotide sequence comprising tandem repeats (e.g., 2-4 repeats) encoding repeating units of the peptide or protein of interest.

In one embodiment, the attenuated bacterium comprises a vector expressing the peptide having the amino acid sequence of SEQ ID NO: 1 or a peptide thereof. In one embodiment, the attenuated bacterium comprises a vector expressing the peptide having the amino acid sequence of SEQ ID NO: 35. In another embodiment, the attenuated bacterium comprises a vector expressing the peptide having the amino acid sequence of SEQ ID NO: 36. In another embodiment, the attenuated bacterium comprises a vector expressing the peptide having the amino acid sequence of SEQ ID NO: 37. In another embodiment, the attenuated bacterium comprises a vector expressing the peptide having the amino acid sequence of SEQ ID NO: 38. In another embodiment, the attenuated bacterium comprises a vector expressing the peptide having the amino acid sequence of SEQ ID NO: 39. In another embodiment, the attenuated bacterium comprises a vector expressing the peptide having the amino acid sequence of SEQ ID NO: 40. In another embodiment, the attenuated bacterium comprises a vector expressing the two or more peptide selected from the peptides of SEQ ID Nos: 35-40.

In another embodiment, the attenuated bacterium comprises a vector expressing the peptide having the amino acid sequence of SEQ ID NO: 41. In another embodiment, the attenuated bacterium comprises a vector expressing the peptide having the amino acid sequence of SEQ ID NO: 42. In another embodiment, the attenuated bacterium comprises a vector expressing the peptide having the amino acid sequence of SEQ ID NO: 43. In another embodiment, the attenuated bacterium comprises a vector expressing the peptide having the amino acid sequence of SEQ ID NO: 44. In another embodiment, the attenuated bacterium comprises a vector expressing the peptide having the amino acid sequence of SEQ ID NO: 45. In another embodiment, the attenuated bacterium comprises a vector expressing the peptide having the amino acid sequence of SEQ ID NO: 46. In another embodiment, the attenuated bacterium comprises a vector expressing the two or more peptide selected from the peptides of SEQ ID Nos: 41-46.

In some embodiments, the attenuated bacterium comprises a vector expressing the peptide having the amino acid sequence of SEQ ID NO: 1 or a peptide thereof, or any of the amino acid sequences of SEQ ID Nos: 35-46 as described above, where the expressed protein or peptide is conjugated to an immunostimulatory peptide which provides a strong T-helper cell (Th) response, including epitopes from potent immunogens such as tetanus toxin, pertussis toxin, the measles virus F protein, and the hepatitis B virus surface antigen (HBsAg), as disclosed in U.S. Pat. No. 5,843,446, which is hereby incorporated by reference. The Th epitopes selected to be conjugated to the protein or peptide thereof as described herein are preferably capable of eliciting T helper cell responses in large numbers of individuals expressing diverse MHC haplotypes. These epitopes function in many different individuals of a heterogeneous population and are considered to be promiscuous Th epitopes. Promiscuous Th epitopes provide an advantage of eliciting potent antibody responses in most members of genetically diverse population groups.

Method of producing the attenuated bacterium transformed with a vector expressing a protein or peptide thereof of SEQ ID NO: 1 can be carried out as described in U.S. Pat. No. 8,685,718 to Wisniewski et al.; Goñi et al., “Mucosal Vaccination Delays or Prevents Prion Infection via an Oral Route,” Neurosci. 133:413-421 (2005); Goñi et al., “High Titers of Mucosal and Systemic Anti-PrP Antibodies Abrogate Oral Prion Infection in Mucosal-Vaccinated Mice,” Neurosci. 153: 679-86 (2008); and Goñi et al., “Mucosal Immunization with an Attenuated Salmonella Vaccine Partially Protects White-Tailed Deer from Chronic Wasting Disease,” Vaccine 33:726-733 (2015), which are hereby incorporated by reference in their entirety, using conventional molecular biology, microbiology, and recombinant DNA techniques within the skill of the art. The general genetic engineering tools and techniques, including transformation and expression, the use of host cells, vectors, expression systems, etc., are well known in the art. See, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. (1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994), which are hereby incorporated by reference in its entirety.

Another aspect of the present disclosure is directed to a vaccine composition comprising the attenuated bacterium as described herein containing a vector expressing a protein or peptide thereof of SEQ ID NO: 1, and a pharmaceutically acceptable carrier.

In one embodiment, the vaccine composition comprises a single attenuated bacterium, where the bacterium is transformed with a vector expressing a protein or SEQ ID NO: 1 or a peptide thereof as described above. In another embodiment, the composition comprises two or more types of attenuated bacteria, each type of attenuated bacterium transformed with a vector expressing a different protein or peptide thereof of SEQ ID NO: 1, e.g., one or more peptides selected from SEQ ID Nos: 35-46. In another embodiment, the composition comprises 2, 3, 4, 5, or 6 types of attenuated bacteria, each type of attenuated bacterium transformed with a vector expressing a different protein or peptide thereof of SEQ ID NO: 1.

A vaccine composition comprising the attenuated bacterium transformed with a vector expressing a protein or peptide as described herein may comprise about 1×10⁹, 1×10¹⁰, 1×10¹¹, 1×10¹², 1×10¹³, 1×10¹⁴, or >1×10¹⁴ CFU/ml in a pharmaceutically acceptable carrier, such as sterile PBS.

The vaccine composition may further comprise one or more adjuvants. In one embodiment, the one or more adjuvant is one that promotes or enhances mucosal immune response. Suitable adjuvants for inclusion in the vaccine compositions described herein include, without limitation, enterotoxin of E. coli; aluminum salts (e.g., aluminum hydroxide that also provides an alkaline environment to facilitate entry into the intestines of the recipient when used as a supplement in an oral immunization); and molecular adjuvants such as CpG DNA, i.e., an oligodeoxynucleotide that has been shown to be an inducer of innate immunity.

The vaccine compositions described herein, whether they comprise the isolated protein, peptide thereof, polymer thereof, or attenuated bacterium expressing the protein or peptide thereof, can be prepared by conventional methods known in the art for the preparation of pharmaceutically acceptable compositions which can be administered to subjects (see e.g., Remington's Pharmaceutical Sciences (Mack Publishing Company, Easton, Pa., USA 1985), which is hereby incorporated by reference in its entirety). The composition can be a liquid solution, suspension, emulsion, tablet, pill, capsule, sustained release formulation, or powder. For oral administration, the vaccine can take the form of, for example, tablets or capsules prepared by conventional means with pharmaceutically acceptable excipients such as binding agents (e.g., pregelatinized maize starch, polyvinyl-pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets can be coated by methods well known in the art.

Preparations for oral administration can also take the form of, for example, solutions, syrups, emulsions or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts (e.g., aluminum hydroxide), flavoring, coloring and sweetening agents as appropriate.

Another aspect of the present disclosure relates to a method of inhibiting the onset of or treat an existing prion disease in a mammalian subject. This method involves administering, to a mammalian subject in need thereof, a vaccine composition as described herein. In one embodiment, the vaccine composition comprises the isolated protein or peptide thereof derived from SEQ ID NO: 1. Exemplary peptides of a vaccine composition include the peptide of SEQ ID Nos: 35-46. As described herein, the isolated protein or peptide thereof may itself be antigenic, may be linked to an adjuvant polypeptide and/or may be encapsulated in a delivery vehicle as described herein. In another embodiment, the vaccine composition comprises one or more polymers of the isolated protein or peptide thereof derived from SEQ ID NO: 1 alone or coupled to an adjuvant polypeptide. Exemplary compositions comprise one or more polymers of a peptide having an amino acid sequence of any one or more of SEQ ID Nos: 35-46. In another embodiment, the vaccine composition comprises one or more attenuated bacteria transformed with a vector expressing one or more isolated proteins or peptides thereof of SEQ ID NO: 1, e.g., any one or more of SEQ ID Nos: 35-46 (alone or coupled to an adjuvant polypeptide). In accordance with this aspect of the disclosure, the vaccine composition is administered to the mammalian subject in an amount effective to inhibit the onset of prion disease is said mammalian subject.

In accordance with this aspect of the disclosure, the vaccine compositions as described herein can be formulated for administration by parenteral, mucosal, topical, intravenous, oral, subcutaneous, intraperitoneal, intranasal or intramuscular means for prophylactic and/or therapeutic treatment. The most typical route of administration is subcutaneous although others can be equally effective. The next most common is intramuscular injection. This type of injection is most typically performed in the arm or leg muscles. Intravenous injections as well as intraperitoneal injections, intra-arterial, intracranial, or intradermal injections are also effective in generating an immune response.

In one embodiment, the vaccine composition is formulated for mucosal delivery, i.e., absorption via the oral, intragastric, intranasal, rectal õr intraocular mucosa to achieve systemic distribution. Methods for mucosal administration of the vaccine compositions as described herein are known in the art, see e.g., U.S. Pat. No. 8,685,718 to Wisniewski, Goñi et la., “Mucosal Immunization with an Attenuated Salmonella Vaccine Partially Protects White-Tailed Deer from Chronic Wasting Disease,” Vaccine 35(5):726-33 (2015), and Goñi et al., “Mucosal Vaccination Delays or Prevents Prior Infection via an Oral Route,” Neuroscience 133(20): 413-21 (2005), which are hereby incorporated by reference in their entirety.

Mammalian subjects which are at risk for exposure to prions or for developing prion disease that are suitable for treatment in accordance with the methods described herein, include, without limitation, humans, deer, elk, cows, sheep, buffalo, bison, moose, hamsters, camel, dromedary, vicuña, alpaca, llama, guanaco, gorilla, chimpanzee, Greater Kudu, wild felines in general, mice, and rats.

As referred to herein, “prion disease” generally refers to a neurodegenerative disease cause by a prion, i.e., a proteinaceous infection particle that causes misfolding of the prion protein. Prion diseases in non-human mammals that can be treated or prevented by employing the methods and compositions described herein include, without limitation, bovine spongiform encephalopathy, chronic wasting disease, scrapie, feline spongiform encephalopathy, transmissible mink encephalopathy, exotic ungulate encephalopathy, or any form of camelid spongiform encephalopathy. Prion diseases in human subjects that can be treated or prevented by employing the methods and compositions described herein include, without limitation, sporadic or familial Creutzfeldt-Jakob disease, variant Creutzfeldt-Jakob disease, Gerstmann-Straussler-Scheinker Syndrome, Fatal Familial Insomnia, and Kuru.

In accordance with the methods described herein, immunotherapy regimens which produce distinct and detectable immune responses following the administration of the fewest number of doses, ideally only one dose, are employed. Specific administration schedules and dosages of the vaccine compositions described herein can be readily determined by the ordinary skilled artisan. The immunization protocol is designed to primarily induce mucosal immunity to the prion protein. The vaccine can be administered in solid form for oral administration or in liquid form. Liquid forms may be administered to a subject either orally or intranasally. For example, unit dosage forms can be administered in intranasal form via topical use of suitable intranasal vehicles.

For example, the vaccines can be mucosally administered as a single dose or divided into multiple doses for administration. In one embodiment, vaccine is administered at least three times at spaced apart intervals, e.g., once weekly, once every 10 days, and once every two weeks. In another embodiment, a single vaccine dose is administered at day 0, and a booster vaccine administered about one week, two weeks, one month, or 2 months later. The booster vaccine can be the same as or different from the initial vaccine composition. For example, in one embodiment, a vaccine composition comprising an attenuated bacterium transformed to express one or more proteins or peptides thereof of SEQ ID NO:1 is initially administered to induce an immune response. The booster vaccine composition that is administered 1, 2, 3, 4, 5, 6, 7, 8, 9, 10, or >10 weeks after the initial vaccine administration is a vaccine composition comprising one or more polymers of the protein and peptides thereof as described herein. In another embodiment, a vaccine composition comprising a cocktail of attenuated bacterium, each transformed to express one or more different proteins or peptides thereof of SEQ ID NO:1 is initially administered to induce an immune response. The booster vaccine composition that is administered 1, 2, 3, 4, 5, 6, 7, 8, 9, 10, or >10 weeks after the initial vaccine administration is a vaccine composition comprising the same or different cocktail of attenuated bacterium, each transformed to express one or more different proteins or peptides thereof of SEQ ID NO:1.

For administration to wild animals such as deer, elk, bison, camels, etc., the vaccine can be mixed with food, and the food placed in a suitable container in an area where the target population lives using bait and baiting systems as have been developed for free-ranging animals for delivery of oral rabies vaccine (where the vaccine is mixed with food the animal likes to eat. (Knobel et al., “Development of a bait and baiting system for delivery of oral rabies vaccine to free-ranging African wild dogs (Lycaon pictus),” J. Wildl. Dis., 38:352-62 (2002), which is hereby incorporated by reference in its entirety.

Another aspect of the present disclosure is directed to animal bait or feed comprising any of the vaccine compositions described herein.

Another aspect of the present disclosure relates to an isolated antibody or epitope-binding portion thereof, wherein the antibody or epitope-binding portion thereof binds to an epitope of a protein or peptide thereof comprising the amino acid sequence of:

(SEQ ID NO: 1)
MX₂X₃X₄X₅X₆GX₈WX₁₀LX₁₂LFVX₁₆X₁₇WX₁₉DX₂₁GX₂₃CKKX₂₇
PKPGGX₃₃WNTGGX₃₉SRYPGQGX₄₇PGGNRYPX₅₅QGGX₅₉X₆₀
WGQPHGGGWGQPHGGX₇₆WGQPHGGX₈₄WGQPHGGX₉₂X₉₃X₉₄
X₉₅X₉₆X₉₇X₉₈X₉₉X₁₀₀GWGQX₁₀₅GX₁₀₇X₁₀₈HX₁₁₀QWX₁₁₃KPX₁₁₆
KPKTX₁₂₁X₁₂₂KHX₁₂₅AGAAAAGAVVGGLGGYX₁₄₂LGSAMSRP
X₁₅₁IHFGX₁₅₆DX₁₅₈EDRYYRENMX₁₆₈RYPX₁₇₂QVYYX₁₇₇PX₁₇₉
X₁₈₀X₁₈₁YX₁₈₃X₁₈₄QNX₁₈₇FVX₁₉₀DCVNITX₁₉₇KX₁₉₉HTVTTTT
KGENFTETDX₂₁₆KX₂₁₈ME X₂₂₁VVEQMCX₂₂₈TQYX₂₃₂X₂₃₃ EX₂₃₅
X₂₃₆ AX₂₃₈X₂₃₉ X₂₄₀ X₂₄₁ X₂₄₂ X₂₄₃ X₂₄₄ X₂₄₅ X₂₄₆ X₂₄₇
X₂₄₈ X₂₄₉ FSX₂₅₂PPVX₂₅₆LLISX₂₆₁LIX₂₆₄ LIVX₂₆₈

wherein X₂ is V or A; X₃ is K or null; X₄ is S or null; X₅ is N or H; X₆ is I, V, L or M; X₈ is S, G, Y or C; X₁₀ is I, L or M; X₁₂ is V, A, or L; X₁₆ is A, T, or V; X₁₇ is T or M; X₁₉ is S or T; X₂₁ is V, I, M; X₂₃ is L or F; X₂₇ is R or W; X₃₃ is G or null; X₃₉ is G or null; X₄₇ S or I; X₅₅ is P or S; X₅₉ is G or null; X₆₀ is G or T; X₇₆ is G or S; X₈₄ is G or S; X₉₂ is G or null; X₉₃ is W or null; X₉₄ is G or null; X₉₅ is Q or null; X % is P or null; X₉₇ is H or null; X₉₈ is G or null; X₉₉ is G or null; X₁₀₀ is G or null; X₁₀₅ is G or S; X₁₀₇ is G or null; X₁₀₈ is T, A, or S; X₁₁₀ is G, N, or S; X₁₁₃ is N or G; X₁₁₆ is S or N; X₁₂₁ is N or S; X₁₁₂ is M or L; X₁₂₅ is V or M; X₁₄₂ is M or L; X₁₅₁ is L, M or I; X₁₅₆ is N or S; X₁₅₈ is Y or W; X₁₆₈ is Y or H; X₁₇₂ is N or E; X₁₇₇ is R or K; X₁₇₉ is V or M; X₁₈₀ is D, N or S; X₁₈₁ is Q, R or E; X₁₈₃ is S or N; X₁₈₄ is N or S; X₁₈₇ is N, S, or T, X₁₉₀ is H or R; X₁₉₇ is V or I; X₁₉₉ is Q or E; X₂₁₆ is V, M or I; X₂₁₈ is M or I; X₂₂₁ is R or Q; X₂₂₈ is I or V; X₂₃₂ is Q, E, or R; X₂₃₃ R, Q, or K; X₂₃₅ is S or Y; X₂₃₆ is Q or E; X₂₃₈ is Y, S, F, or A; X₂₃₉ is Y or Q; X₂₄₀ is Q, D, or G; X₂₄₁ is G or null; X₂₄₂ R or null; X₂₄₃ is R or K; X₂₄₄ is G, S, or A; X₂₄₅ is A or S; X₂₄₆ is S or G; X₂₄₇ is V, T, A, M or null; X₂₄₈ is I, L, V; X₂₄₉ is L or I; X₂₅₂ is S or P; X₂₅₆ is I or V; X₂₆₁ is F or L; X₂₆₄ is F or L; X₂₆₈ is G or R; and wherein the protein or peptide thereof does not comprise an amino acid sequence of a corresponding protein of any one of SEQ ID Nos: 2-34 or a corresponding peptide thereof.

In one embodiment, the antibody or epitope-binding portion thereof is raised against the protein or peptide thereof of SEQ ID NO: 1.

Another aspect of the present disclosure relates to an isolated antibody or epitope-binding portion thereof, wherein said antibody or epitope-binding portion thereof binds to a polymer of a protein or peptide thereof, wherein said protein or peptide thereof of the polymer comprises the amino acid sequence of:

(SEQ ID NO: 1)
MX₂X₃X₄X₅X₆GX₈WX₁₀LX₁₂LFVX₁₆X₁₇WX₁₉DX₂₁GX₂₃CKKX₂₇
PKPGGX₃₃WNTGGX₃₉SRYPGQGX₄₇PGGNRYPX₅₅QGGX₅₉X₆₀
WGQPHGGGWGQPHGGX₇₆WGQPHGGX₈₄WGQPHGGX₉₂X₉₃X₉₄
X₉₅X₉₆X₉₇X₉₈X₉₉X₁₀₀GWGQX₁₀₅GX₁₀₇X₁₀₈HX₁₁₀QWX₁₁₃KPX₁₁₆
KPKTX₁₂₁X₁₂₂KHX₁₂₅AGAAAAGAVVGGLGGYX₁₄₂LGSAMSRP
X₁₅₁IHFGX₁₅₆DX₁₅₈EDRYYRENMX₁₆₈RYPX₁₇₂QVYYX₁₇₇PX₁₇₉
X₁₈₀X₁₈₁YX₁₈₃X₁₈₄QNX₁₈₇FVX₁₉₀DCVNITX₁₉₇KX₁₉₉HTVTTTT
KGENFTETDX₂₁₆KX₂₁₈ME X₂₂₁VVEQMCX₂₂₈TQYX₂₃₂X₂₃₃ EX₂₃₅
X₂₃₆ AX₂₃₈X₂₃₉ X₂₄₀ X₂₄₁ X₂₄₂ X₂₄₃ X₂₄₄ X₂₄₅ X₂₄₆ X₂₄₇
X₂₄₈ X₂₄₉ FSX₂₅₂PPVX₂₅₆LLISX₂₆₁LIX₂₆₄ LIVX₂₆₈

wherein X₂ is V or A; X₃ is K or null; X₄ is S or null; X₅ is N or H; X₆ is I, V, L or M; X₈ is S, G, Y or C; X₁₀ is I, L or M; X₁₂ is V, A, or L; X₁₆ is A, T, or V; X₁₇ is T or M; X₁₉ is S or T; X₂₁ is V, I, M; X₂₃ is L or F; X₂₇ is R or W; X₃₃ is G or null; X₃₉ is G or null; X₄₇ S or I; X₅₅ is P or S; X₅₉ is G or null; X₆₀ is G or T; X₇₆ is G or S; X₈₄ is G or S; X₉₂ is G or null; X₉₃ is W or null; X₉₄ is G or null; X₉₅ is Q or null; X₉₆ is P or null; X₉₇ is H or null; X₉₈ is G or null; X₉₉ is G or null; X₁₀₀ is G or null; X₁₀₅ is G or S; X₁₀₇ is G or null; X₁₀₈ is T, A, or S; X₁₁₀ is G, N, or S; X₁₁₃ is N or G; X₁₁₆ is S or N; X₁₂₁ is N or S; X₁₂₂ is M or L; X₁₂₅ is V or M; X₁₄₂ is M or L; X₁₅₁ is L, M or I; X₁₅₆ is N or S; X₁₅₈ is Y or W; X₁₆₈ is Y or H; X₁₇₂ is N or E; X₁₇₇ is R or K; X₁₇₉ is V or M; X₁₈₀ is D, N or S; X₁₈₁ s is Q, R or E; X₁₈₃ is S or N; X₁₈₄ is N or S; X₁₈₇ is N, S, or T, X₁₉₀ is H or R; X₁₉₇ is V or I; X₁₉₉ is Q or E; X₂₁₆ is V, M or I; X₂₁₈ is M or I; X₂₂₁ is R or Q; X₂₂₈ is I or V; X₂₃₂ is Q, E, or R; X₂₃₃ R, Q, or K; X₂₃₅ is S or Y; X₂₃₆ is Q or E; X₂₃₈ is Y, S, F, or A; X₂₃₉ is Y or Q; X₂₄₀ is Q, D, or G; X₂₄₁ is G or null; X₂₄₂ R or null; X₂₄₃ is R or K; X₂₄₄ is G, S, or A; X₂₄₅ is A or S; X₂₄₆ is S or G; X₂₄₇ is V, T, A, M or null; X₂₄₈ is I, L, V; X₂₄₉ is L or I; X₂₅₂ is S or P; X₂₅₆ is I or V; X₂₆₁ is F or L; X₂₆₄ is F or L; X₂₆₈ is G or R; and

wherein the protein or peptide thereof does not comprise an amino acid sequence of a corresponding protein of any one of SEQ ID Nos: 2-34 or a corresponding peptide thereof.

In one embodiment, the antibody or epitope-binding portion thereof is raised against a polymer of a protein or peptide thereof of SEQ ID NO: 1.

As used herein, “epitope” refers to the antigenic determinant of the isolated protein or peptide thereof that is recognized by the isolated antibody. The epitope recognized by the antibody of the present invention may be a linear epitope, i.e. the primary structure of the amino acid sequence of the isolated protein or peptide thereof. Preferably, the linear epitope recognized by the isolated antibody of the present invention comprises a non-naturally occurring amino acid sequence, i.e., is not a sequence found within any of SEQ ID Nos: 2-34. Alternatively, the epitope recognized by the isolated antibody or epitope binding portion thereof is a non-linear or conformational epitope, i.e. the tertiary or quaternary structure of a polymerized protein or peptide thereof as described herein. More preferably, the non-linear or conformational epitope recognized by the isolated antibody or epitope-binding portion thereof is a conformational epitope that is common or shared with one or more, or all, prion proteins. Accordingly, the isolated antibody of the present invention has antigenic specificity for a shared conformational epitope common to all pathological prion proteins known in the art.

An isolated antibody of the present disclosure encompasses any immunoglobulin molecule that specifically binds to a linear or conformational epitope of an isolated protein or peptide thereof of SEQ ID NO: 1 as defined herein. As used herein, the term “antibody” is meant to include intact immunoglobulins derived from natural sources or from recombinant sources, as well as immunoreactive portions (i.e., antigen binding portions) of intact immunoglobulins. The antibodies of the present invention may exist in a variety of forms including, for example, polyclonal antibodies, monoclonal antibodies, intracellular antibodies (“intrabodies”), antibody fragments (e.g. Fv, Fab and F(ab)2), as well as single chain antibodies (scFv), chimeric antibodies and humanized antibodies (Ed Harlow and David Lane, USING ANTIBODIES: A LABORATORY MANUAL (Cold Spring Harbor Laboratory Press, 1999); Houston et al., “Protein Engineering of Antibody Binding Sites: Recovery of Specific Activity in an Anti-Digoxin Single-Chain Fv Analogue Produced in Escherichia coli,” Proc NatlAcadSci USA 85:5879-5883 (1988); Bird et al, “Single-Chain Antigen-Binding Proteins,” Science 242:423-426 (1988)) or VHH camelid antibodies.

Antibodies of the present disclosure may also be synthetic antibodies. A synthetic antibody is an antibody which is generated using recombinant DNA technology, such as, for example, an antibody expressed by a bacteriophage. Alternatively, the synthetic antibody is generated by the synthesis of a DNA molecule encoding and expressing the antibody of the invention or the synthesis of an amino acid specifying the antibody, where the DNA or amino acid sequence has been obtained using synthetic DNA or amino acid sequence technology which is available and well known in the art.

Methods for monoclonal antibody production may be carried out using the techniques described herein or other well-known in the art (MONOCLONAL ANTIBODIES—PRODUCTION, ENGINEERING AND CLINICAL APPLICATIONS (Mary A. Ritter and Heather M. Ladyman eds., 1995), which is hereby incorporated by reference in its entirety). Generally, the process involves obtaining immune cells (lymphocytes) from the spleen of a mammal which has been previously immunized with the antigen of interest (e.g., a polymer of a protein or peptide described herein or an attenuated bacterium transformed with a vector expressing the protein or peptide thereof) either in vivo or in vitro. Exemplary peptides (e.g., SEQ ID Nos: 35-40 and 41-46) are described supra.

The antibody-secreting lymphocytes are then fused with myeloma cells or transformed cells, which are capable of replicating indefinitely in cell culture, thereby producing an immortal, immunoglobulin-secreting cell line. Fusion with mammalian myeloma cells or other fusion partners capable of replicating indefinitely in cell culture is achieved by standard and well-known techniques, for example, by using polyethylene glycol (PEG) or other fusing agents (Milstein and Kohler, “Derivation of Specific Antibody-Producing Tissue Culture and Tumor Lines by Cell Fusion,” Eur J Immunol 6:511 (1976), which is hereby incorporated by reference in its entirety). The immortal cell line, which is preferably murine, but may also be derived from cells of other mammalian species, is selected to be deficient in enzymes necessary for the utilization of certain nutrients, to be capable of rapid growth, and have good fusion capability. The resulting fused cells, or hybridomas, are cultured, and the resulting colonies screened for the production of the desired monoclonal antibodies. Colonies producing such antibodies are cloned, and grown either in vivo or in vitro to produce large quantities of antibody.

Alternatively, the antibodies described herein can be prepared by any of a variety recombinant DNA techniques using isolated polynucleotides, vectors, and host cells. In general, antibodies can be produced by cell culture techniques, including the generation of monoclonal antibodies via conventional techniques, or via transfection of antibody genes, heavy chains and/or light chains into suitable bacterial or mammalian cell hosts, in order to allow for the production of antibodies, wherein the antibodies may be recombinant. Standard molecular biology techniques are used to prepare the recombinant expression vector, transfect the host cells, select for transformants, culture the host cells and recover the antibody from the culture medium. Transfecting the host cell can be carried out using a variety of techniques commonly used for the introduction of exogenous DNA into a prokaryotic or eukaryotic host cell, e.g., by electroporation, calcium-phosphate precipitation, DEAE-dextran transfection and the like. Although it is possible to express the antibodies described herein in either prokaryotic or eukaryotic host cells, expression of antibodies in eukaryotic cells is sometimes preferable, and sometimes preferable in mammalian host cells, because such eukaryotic cells (and in particular mammalian cells) are more likely than prokaryotic cells to assemble and secrete a properly folded and immunologically active antibody.

As noted above, exemplary mammalian host cells for expressing the recombinant antibodies of the invention include Chinese Hamster Ovary (CHO cells) (including dhfr-CHO cells, described in Urlaub and Chasin, Proc. Natl. Acad. Sci. USA, 77: 4216-4220 (1980), which is hereby incorporated by reference in its entirety). Other suitable mammalian host cells include, without limitation, NS0 myeloma cells, COS cells, and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown.

Host cells can also be used to produce functional antibody fragments, such as Fab fragments or scFv molecules. It is understood that variations on the above procedure are within the scope of the present disclosure. For example, it may be desirable to transfect a host cell with DNA encoding functional fragments of either the light chain and/or the heavy chain of an antibody described herein. Recombinant DNA technology may also be used to remove some or all the DNA encoding either or both of the light and heavy chains that is not necessary for binding to the antigens of interest. The molecules expressed from such truncated DNA molecules are also encompassed by the antibodies described herein.

In one embodiment, the sequence of the polynucleotide molecules encoding the antibodies and binding fragments or the proteins and peptides thereof of SEQ ID NO: 1 described herein are modified using gene editing technology. Suitable gene editing technology and systems include, for example, zinc finger nucleases (“ZFNs”) (Urnov et al., “Genome Editing with Engineered Zinc Finger Nucleases,” Nat. Rev. Genet. 11: 636-646 (2010), which is hereby incorporated by reference in its entirety), transcription activator-like effector nucleases (“TALENs”) (Joung & Sander, “TALENs: A Widely Applicable Technology for Targeted Genome Editing,” Nat. Rev. Mol. Cell Biol. 14: 49-55 (2013), which is hereby incorporated by reference in its entirety), clustered regularly interspaced short palindromic repeat (“CRISPR”)-associated endonucleases (e.g., CRISPR/CRISPR-associated (“Cas”) 9 systems) (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety). Gene editing modifications can be employed for humanization, class-switch recombination, and/or antibody fragment production (see e.g., Cheong et al., “Editing of Mouse and Human Immunoglobulin Genes by CRISPR-Cas9 System,” Nature Comm. 7: 10934 (2016), and Flisikowska et al., “Efficient Immunoglobulin Gene Disruption and Targeted Replacement in Rabbit Using Zinc Finger Nucleases,” PLOS One 6(6): e21045 (2011), which are hereby incorporated by reference in their entirety.

The antibodies and antibody binding fragments are recovered and purified from recombinant cell cultures by known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be used for purification.

The monoclonal antibody of the present disclosure can be a humanized antibody. Humanized antibodies are antibodies that contain minimal sequences from non-human (e.g., murine) antibodies within the variable regions. Such antibodies are used therapeutically to reduce antigenicity and human anti-mouse antibody responses when administered to a human subject. In practice, humanized antibodies are typically human antibodies with minimal to no non-human sequences. A human antibody is an antibody produced by a human or an antibody having an amino acid sequence corresponding to an antibody produced by a human.

An antibody can be humanized by substituting the complementarity determining region (CDR) of a human antibody with that of a non-human antibody (e.g., mouse, rat, rabbit, hamster, etc.) having the desired specificity, affinity, and capability (Jones et al., “Replacing the Complementarity-Determining Regions in a Human Antibody With Those From a Mouse,” Nature 321:522-525 (1986); Riechmann et al., “Reshaping Human Antibodies for Therapy,” Nature 332:323-327 (1988); Verhoeyen et al., “Reshaping Human Antibodies: Grafting an Antilysozyme Activity,” Science 239:1534-1536 (1988), which are hereby incorporated by reference in their entirety). The humanized antibody can be further modified by the substitution of additional residues either in the Fv framework region and/or within the replaced non-human residues to refine and optimize antibody specificity, affinity, and/or capability.

Human antibodies can be produced using various techniques known in the art. Immortalized human B lymphocytes immunized in vitro or isolated from an immunized individual that produce an antibody directed against a target antigen can be generated (See e.g., Reisfeld et al., MONOCLONAL ANTIBODIES AND CANCER THERAPY 77 (Alan R. Liss ed., 1985) and U.S. Pat. No. 5,750,373 to Garrard, which are hereby incorporated by reference in their entirety). Also, the human antibody can be selected from a phage library, where that phage library expresses human antibodies (Vaughan et al., “Human Antibodies with Sub-Nanomolar Affinities Isolated from a Large Non-immunized Phage Display Library,” Nature Biotechnology, 14:309-314 (1996); Sheets et al., “Efficient Construction of a Large Nonimmune Phage Antibody Library: The Production of High-Affinity Human Single-Chain Antibodies to Protein Antigens,” Proc. Natl. Acad. Sci. U.S.A. 95:6157-6162 (1998); Hoogenboom et al., “By-passing Immunization. Human Antibodies From Synthetic Repertoires of Germline VH Gene Segments Rearranged In Vitro,” J Mol Biol 227:381-8 (1992); Marks et al., “By-passing Immunization. Human Antibodies from V-gene Libraries Displayed on Phage,” J Mol Biol 222:581-97 (1991), which are hereby incorporated by reference in their entirety). Human antibodies can also be made in transgenic mice containing human immunoglobulin loci that are capable upon immunization of producing the full repertoire of human antibodies in the absence of endogenous immunoglobulin production. This approach is described in U.S. Pat. No. 5,545,807 to Surani et al.; U.S. Pat. No. 5,545,806 to Lonberg et al.; U.S. Pat. No. 5,569,825 to Lonberg et al.; U.S. Pat. No. 5,625,126 to Lonberg et al.; U.S. Pat. No. 5,633,425 to Lonberg et al.; and 5,661,016 to Lonberg et al., which are hereby incorporated by reference in their entirety.

Procedures for raising polyclonal antibodies are also well known. Typically, such antibodies can be raised by administering the isolated protein or peptide thereof as described herein or a polymer of such isolated protein or peptide thereof as described herein subcutaneously to rabbits (e.g., New Zealand white rabbits), goats, sheep, swine, or donkeys which have been bled to obtain pre-immune serum. The antigens can be injected in combination with an adjuvant. The rabbits are bled approximately every two weeks after the first injection and periodically boosted with the same antigen three times every six weeks. Polyclonal antibodies are recovered from the serum by affinity chromatography using the corresponding antigen to capture the antibody. This and other procedures for raising polyclonal antibodies are disclosed in Ed Harlow and David Lane, USING ANTIBODIES: A LABORATORY MANUAL (Cold Spring Harbor Laboratory Press, 1988), which is hereby incorporated by reference in its entirety.

In addition to whole antibodies, the present invention encompasses binding portions of such antibodies. Such binding portions include the monovalent Fab fragments, Fv fragments (e.g., single-chain antibody, scFv), and single variable V_{H} and V_{L} domains, and the bivalent F(ab′)₂ fragments, Bis-scFv, diabodies, triabodies, minibodies, etc. These antibody fragments can be made by conventional procedures, such as proteolytic fragmentation procedures, as described in James Goding, MONOCLONAL ANTIBODIES: PRINCIPLES AND PRACTICE 98-118 (Academic Press, 1983) and Ed Harlow and David Lane, ANTIBODIES: A LABORATORY MANUAL (Cold Spring Harbor Laboratory, 1988), which are hereby incorporated by reference in their entirety, or other methods known in the art.

Also suitable for use in the present invention are antibody fragments engineered to bind to intracellular proteins, i.e. intrabodies. Accordingly, an intrabody can be used to bind selectively to an epitope of a pathological prion protein within a cell. Intrabodies are generally obtained by selecting a single variable domain from variable regions of an antibody having two variable domains (i.e., a heterodimer of a heavy chain variable domain and a light chain variable domain). Single chain Fv fragments, Fab fragments, ScFv-Ck fusion proteins, single chain diabodies, V_{H}-C_{H}1 fragments, and even whole IgG molecules are suitable formats for intrabody development (Kontermann R. E., “Intrabodies as Therapeutic Agents,” Methods 34:163-70 (2004), which is here by incorporated by reference in its entirety).

Intrabodies having antigen specificity for a conformational epitope of an prion protein can be obtained from phage display, yeast surface display, or ribosome surface display. Methods for producing libraries of intrabodies and isolating intrabodies of interest are further described in U.S. Published Patent Application No. 20030104402 to Zauderer and U.S. Published Patent Application No. 20050276800 to Rabbitts, which are hereby incorporated by reference in their entirety. Methods for improving the stability and affinity binding characteristics of intrabodies are described in WO2008070363 to Zhenping; Contreras-Martinez et al., “Intracellular Ribosome Display via SecM Translation Arrest as a Selection for Antibodies with Enhanced Cytosolic Stability,” J Mol Biol 372(2):513-24 (2007), which are hereby incorporated by reference in their entirety.

It may further be desirable, especially in the case of antibody fragments, to modify the antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Antibody mimics are also suitable for use in accordance with the present invention. A number of antibody mimics are known in the art including, without limitation, those known as monobodies, which are derived from the tenth human fibronectin type III domain (¹⁰Fn3) (Koide et al., “The Fibronectin Type III Domain as a Scaffold for Novel Binding Proteins,” J Mol Biol 284:1141-1151 (1998); Koide et al., “Probing Protein Conformational Changes in Living Cells by Using Designer Binding Proteins: Application to the Estrogen Receptor,” Proc Natl Acad Sci USA 99:1253-1258 (2002), each of which is hereby incorporated by reference in its entirety); and those known as affibodies, which are derived from the stable alpha-helical bacterial receptor domain Z of staphylococcal protein A (Nord et al., “Binding Proteins Selected from Combinatorial Libraries of an alpha-helical Bacterial Receptor Domain,” Nature Biotechnol 15(8):772-777 (1997), which is hereby incorporated by reference in its entirety).

Another aspect of the present disclosure is directed to a pharmaceutical composition comprising an antibody or epitope-binding portion thereof and a pharmaceutically acceptable carrier.

Other aspects of the present disclosure relate to methods of preventing the onset of a prion disease in a mammalian subject and method of treating a mammalian subject having a prion disease. This method involves administering to the mammalian subject an antibody or epitope-binding portion or a pharmaceutical composition containing the same as described herein. In one embodiment, the subject is at risk for acquiring a prion disease and is thus, administered the antibody or epitope-binding portion thereof or a pharmaceutical composition containing the same in an amount effective to prevent the onset of the prion disease. In another embodiment, the subject has prion disease, and, thus, is administered the antibody or epitope-binding portion thereof or a pharmaceutical composition containing the same in an amount effective to treat the prion disease in the subject. In accordance with this aspect of the invention, the antibody or pharmaceutical composition containing the antibody is administered in an amount effective to generate passive immunity in the mammalian subject against one or more pathological prion proteins, thereby facilitating the clearance of the pathological prion protein from the subject. Suitable mammalian subjects and prion conditions that can be treated in accordance with this aspect of the present disclosure are described supra.

For passive immunization with a composition comprising an antibody or epitope-binding portion thereof as described herein, the dosage ranges from about 0.0001 to 100 mg/kg, and more usually 0.01 to 5 mg/kg of the host body weight. An exemplary treatment regime entails administration once per every two weeks or once a month or once every 3 to 6 months. In some methods, two or more monoclonal antibodies with different binding specificities are administered simultaneously, in which case the dosage of each antibody administered falls within the ranges indicated. Antibody is usually administered on multiple occasions. Intervals between single dosages can be weekly, monthly or yearly. Intervals can also be irregular as indicated by measuring blood levels of antibody to polymerized first or second peptides or fusion peptides in the patient. Alternatively, antibody can be administered as a sustained release formulation, in which case less frequent administration is required. Dosage and frequency vary depending on the half-life of the antibody in the patient. In general, human antibodies show the longest half life, followed by humanized antibodies, chimeric antibodies, and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### Materials and Methods for Example 1

PRNP Sequencing. Genomic DNA was purified from 200 μL of whole blood using commercial kits (Illustra G&E, or Purelink Genomic DNA mini spin kit, Invitrogen). Polymerase chain reaction (PCR) was performed with forward primer: 5′-CACAGCAGATATAAGTCATCATGGTG-3′ (SEQ ID NO:53) and reverse primer 5′-GGTAAAGATGATTAAGAAGATGATGAAAACAGGAAGG-3′ (SEQ ID NO:54). These primers amplify the Codificant region of the PRNP gene and were designed based on PRNP gene sequences from different species available in GenBank. PCR was carried out in 50 μl reaction mixtures containing 50 ng of purified DNA, 0.2 μM of each dNTP, 15 pmol of each primer, 20 mM Tris-HCl, pH 8.4, 50 mM KCl, 3.0 mM MgCl₂, and 2.5 U of Taq DNA Polymerase (Invitrogen). Cycling conditions after initial denaturation at 94° C. for 3 minutes were 35 cycles of denaturation (94° C., 30 seconds), annealing (48° C., 30 seconds) and extension (72° C., 45 seconds), and a final extension at 72° C. for 5 minutes. PCR fragments (˜850 bp) were purified using Pure link PCR purification kit (Invitrogen) and sequenced directly (using the primers described above) or cloned into pGEM-T easy vector (Promega) and then sequenced (using primers sp6 and t7). Sequencing was carried out using an 3500×1 Genetic Analyzer (Applied Biosystems) by INTA's Genomic Unit (Biotechnology Institute, INTA, Buenos Aires, Argentina).

The nucleotide sequences of newly sequenced PRNP genes are set forth below.

777 deer (white tailed) odocoileus 96S (SEQ ID NO: 55):
MVKSHIGSWILVLFVAMWSDVGLCKKRPKPGGGWNTGGSRYPGQGSPGGNRYPPQ
GGGGWGQPHGGGWGQPHGGGWGQPHGGGWGQPHGGGGWGQSGTHSQWNKPSK
PKTNMKHVAGAAAAGAVVGGLGGYMLGSAMSRPLIHFGNDYEDRYYRENMYRYP
NQVYYRPVDQYNNQNTFVHDCVNITVKQHTVTTTTKGENFTETDIKMMERVVEQM
CITQYQRESQAYYQRGASVILFSSPPVILLISFLIFLIVG
781 deer (white tailed) odocouileus 103N (SEQ ID NO: 56):
MVKSHIGSWILVLFVAMWSDVGLCKKRPKPGGGWNTGGSRYPGQGSPGGNRYPPQ
GGGGWGQPHGGGWGQPHGGGWGQPHGGGWGQPHGGGGWGQGGTHSQWNKPS
KPKTNMKHVAGAAAAGAVVGGLGGYMLGSAMSRPLIHFGNDYEDRYYRENMYRY
PNQVYYRPVDQYNNQNTFVHDCVNITVKQHTVTTTTKGENFTETDIKMMERVVEQ
MCITQYQRESQAYYQRGASVILFSSPPVILLISFLIFLIVG
99 red deer (SEQ ID NO: 57):
MVKSHIGSWILVLFVAMWSDVGLCKKRPKPGGGWNTGGSRYPGQGSPGGNRYPPQ
GGGGWGQPHGGGWGQPHGGGWGQPHGGGWGQPHGGGGWGQGGAHSQWNKPS
KPKTNMKHVAGAAAAGAVVGGLGGYMLGSAMSRPLIHFGNDYEDRYYRENMYRY
PNQVYYRPVDQYNNQNTFVHDCVNITVKQHTVTTTTKGENFTETDIKMMERVVEQ
MCITQYQRESQAYYQRGASVILFSSPPVILLISFLIFLIVR
WATER BUFFALO 2CTL (SEQ ID NO: 58):
MVKSHIGSWILVLFVVMWSDVGLCKKRPKPGGGWNTGGSRYPGQGSPGGNRYPSQ
GGGGWGQPHGGGWGQPHGGGWGQPHGGGWGQPHGGGWGQPHGGGGWGQGGT
HGQWNKPSKPKTNMKHMAGAAAAGAVVGGLGGYMLGSAMSRPLIHFGNDYEDR
YYRENMHRYPNQVYYRPVDQYSNQNNFVHDCVNITVKEHTVTTTTKGENFTETDIK
MMERVVEQMCITQYQRESQAYYQRGASVILFSSPPVILLISLLIFLIVR
WATER BUFFALO 19 (SEQ ID NO: 59):
MVKSHIGSWILVLFVVMWSDVGLCKKRPKPGGGWNTGGSRYPGQGSPGGNRYPSQ
GGGGWGQPHGGGWGQPHGGGWGQPHGGGWGQPHGGGWGQPHGGGGWGQGGT
HSQWNKPSKPKTNMKHMAGAAAAGAVVGGLGGYMLGSAMSRPLIHFGNDYEDRY
YRENMHRYPNQVYYRPVDQYSNQNNFVHDCVNITVKEHTVTTTTKGENFTETDIK
MMERVVEQMCITQYQRESQAYYQRGASVILFSSPPVILLISLLIFLIVR
alpaca (SEQ ID NO: 60):
MVKSHMGSWILVLFVVTWSDMGLCKKRPKPGGGWNTGGSRYPGQGSPGGNRYPP
QGGGGWGQPHGGGWGQPHGGGWGQPHGGGWGQPHGGGWGQGGGTHGQWNKP
SKPKTSMKHVAGAAAAGAVVGGLGGYMLGSAMSRPLIHFGNDYEDRYYRENMYR
YPNQVYYKPVDQYSNQNSFVHDCVNITVKQHTVTTTTKGENFTETDVKMMERVVE
QMCITQYQREYQASYGRGASVIFSSPPVILLISFLIFLIVG
Guanaco-DB6D (SEQ ID NO: 61):
MVKSHMGSWILVLFVVTWSDMGLCKKRPKPGGGWNTGGSRYPGQGSPGGNRYPP
QGGGGWGQPHGGGWGQPHGGGWGQPHGGGWGQPHGGGWGQGGGTHGQWNKP
SKPKTSMKHVAGAAAAGAVVGGLGGYMLGSAMSRPLIHFGNDYEDRYYRENMYR
YPNQVYYKPVDQYSNQNSFVHDCVNITVKQHTVTTTTKGENFTETDVKMMERVVE
QMCITQYQREYQASYGRGASVIFSSPPVILLISFLI
vicuilia 351E (SEQ ID NO: 62):
MVKSHIGSWILVLFVVTWSDMGLCKKRPKPGGGWNTGGSRYPGQGSPGGNRYPPQ
GGGGWGQPHGGGWGQPHGGGWGQPHGGGWGQPHGGGWGQGGGTHGQWNKPS
KPKTSMKHVAGAAAAGAVVGGLGGYMLGSAMSRPLIHFGNDYEDRYYRENMYRY
PNQVYYKPVDQYSNQNSFVHDCVNITVKQHTVTTTTKGENFTETDVKMMERVVEQ
MCITQYQREYQASYGRGASVIFSSPPVILLISFLIFLIV
llama 77DC (SEQ ID NO: 63):
MVKSHIGSWILVLFVVTWSDVGLCKKRPKPGGGWNTGGSRYPGQGSPGGNRYPPQ
GGGGWGQPHGGGWGQPHGGGWGQPHGGGWGQPHGGGWGQGGGTHGQWNKPS
KPKTSMKHVAGAAAAGAVVGGLGGYMLGSAMSRPLIHFGNDYEDRYYRENMYRY
PNQVYYKPVDQYSNQNSFVHDCVNITVKQHTVTTTTKGENFTETDVKMMERVVEQ
MCITQYQREYQASYGRGASVIFSSPPVILLISFLIFLIV

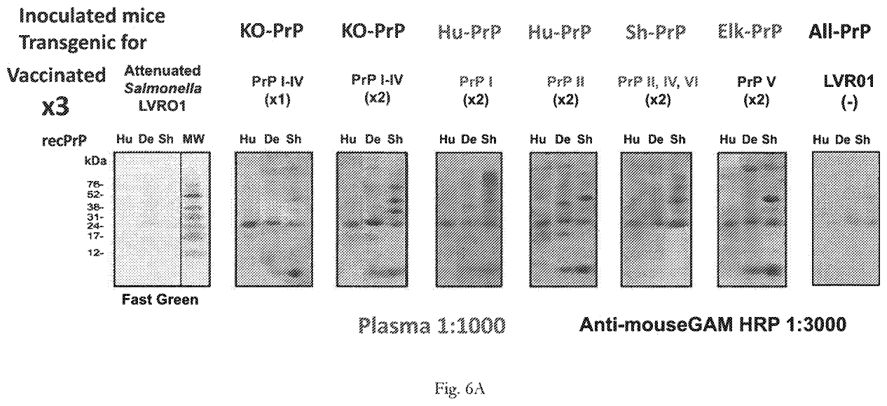

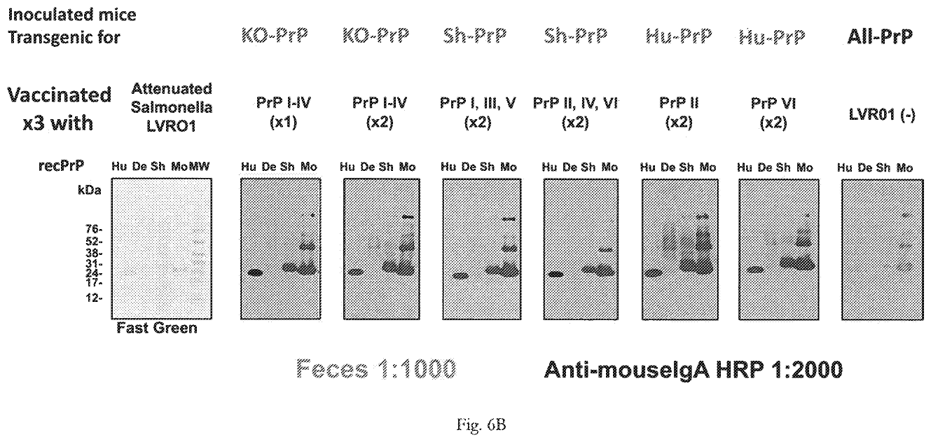

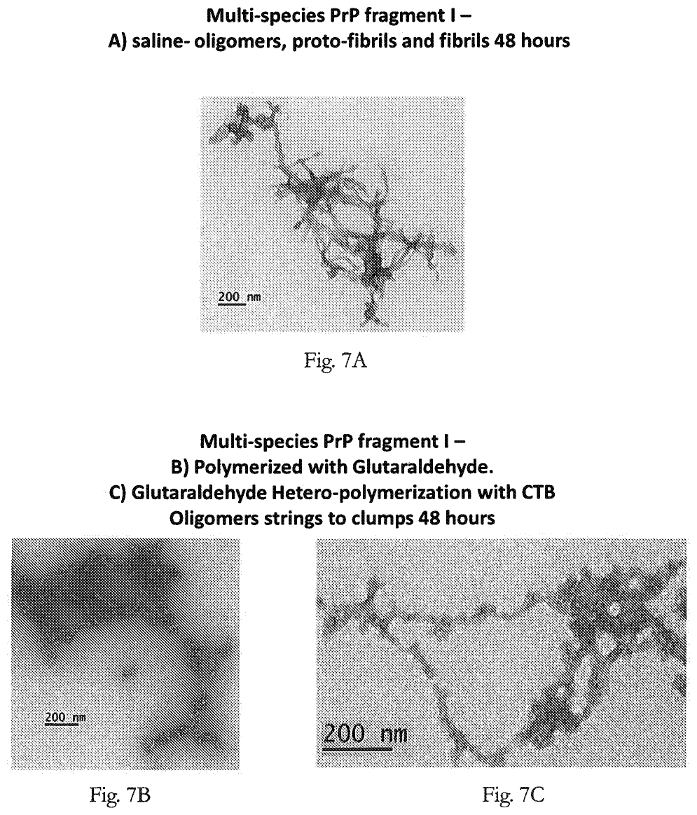

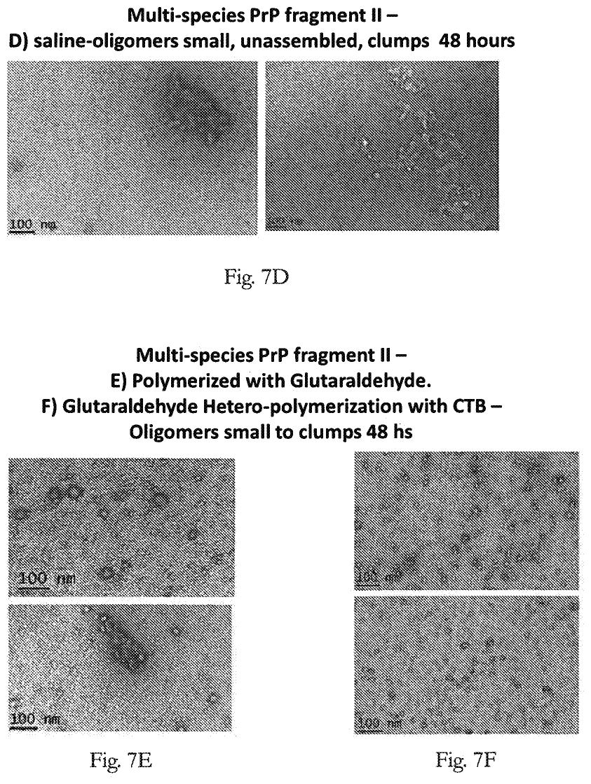

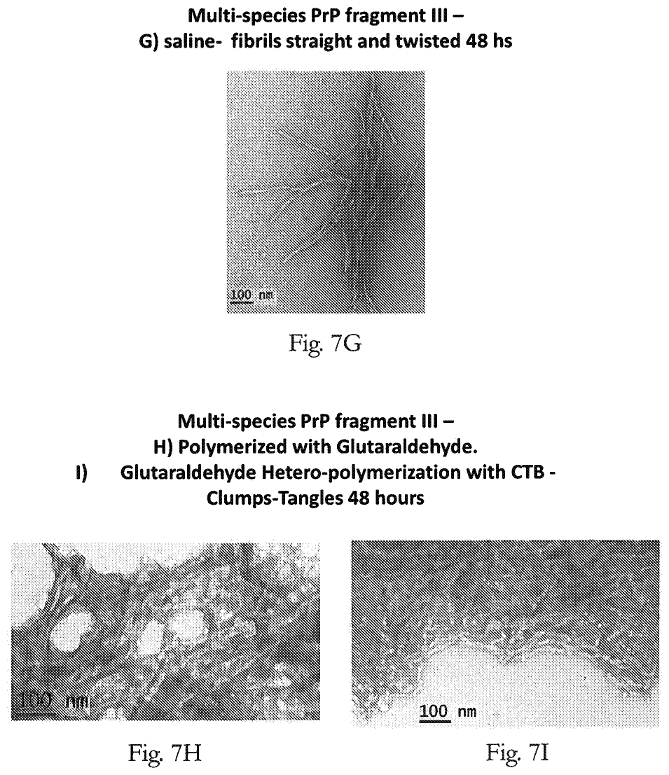

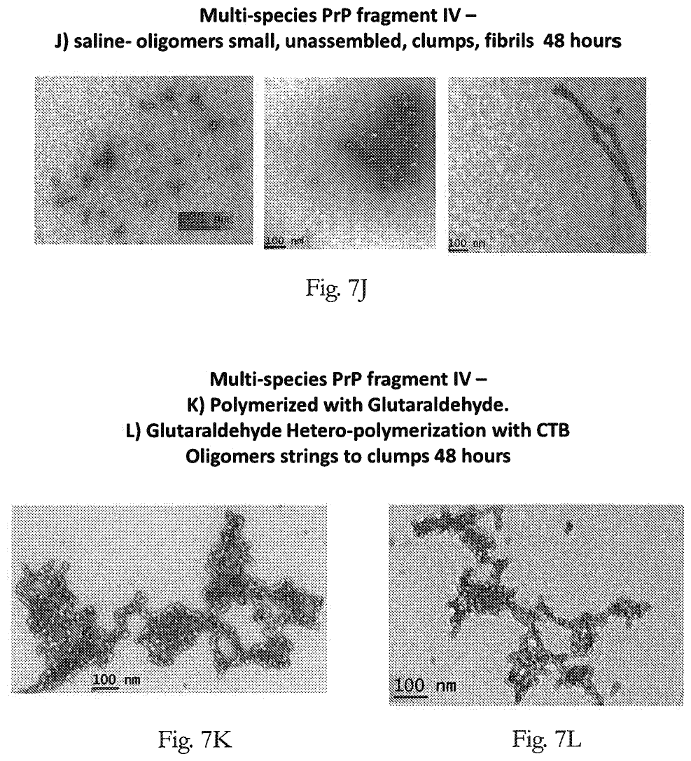

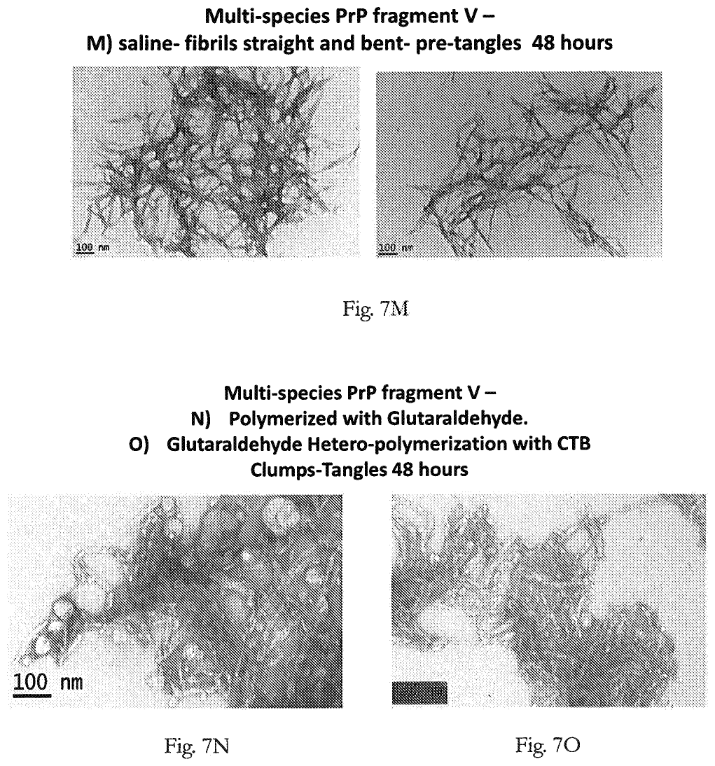

Peptide Synthesis and Polymerization. The peptides shown in Table 1 were synthesized and used for Electron Microscopy in saline, after controlled polymerization with glutaraldehyde (using the procedure outlined below), and after hetero-polymerization with Cholera Toxin B (CTB) on a controlled reaction with glutaraldehyde (FIGS. 7A-7O). The results demonstrate that some of the peptides produce fibrils after 48 hs in saline, while when polymerized with glutaraldehyde and after hetero-polymerization with CTB, they produced oligomers, and tangles, clumps. Thus, the peptides polymerized or hetero-polymerized with CTB on controlled reaction with glutaraldehyde were deemed to be safe for inoculation, without possibility of producing pathologic fibril seeds. The peptides were inoculated in mice as second boost after the original attenuated Salmonella immunization with the corresponding peptides. The results of the original Salmonella with the multi-species PrP peptides I-VI immunizations and the polymerized PrP I-VI peptides boosts are shown on FIGS. 6A and 6B. All PrP multi-species I-VI produced antibodies (IgG, IgM, IgA in plasma and secretory IgA in feces) that recognized monomeric and oligomeric species on samples from purified pathological PrP prions from different species human, deer and sheep (FIGS. 6A, 6B). Sequence adapters (shown in bold underline in Table 1) were added at the amino and carboxyl ends to assure linker flexibility and proper secondary structure conformation of the PrP amino acids and the potential new epitopes.

TABLE 1
Synthesized Peptide Sequences

| | | | Corresponding |
|---|---|---|---|
| | | SEQ | Amino Acids |
| Peptide | | ID | of SEQ ID |
| Name | Synthesized Peptide Sequence | NO: | NO: 1 |
| PrP I | [symbol] RYPPQGGGWGQPHGGGWGQPHGG [symbol] | 64 | 52-75 |
| PrP II | [symbol] QGGSHSQWNKPSKPKTNLKHMAGAAAAGAV | 65 | 104-134 |
| PrP III | [symbol] GAVVGGLGGYMLGSAMSRPIIHFGNDWE [symbol] | 66 | 132-159 |
| PrP IV | [symbol] GSDYEDRYYRENMYRYPEQVYYRPMDRY [symbol] | 67 | 155-182 |
| PrP V | [symbol] YRPMDEYSSQNSFVRDCVNITIKQHTVTT | 68 | 176-204 |
| PrP VI | [symbol] TTTTKGENFTETDIKMMERVVEQMCVTQYER | 69 | 203-233 |

The peptides were subjected to controlled polymerization using the following protocol. The peptides were dissolved at 3 mg/ml, in 100 mM borate buffer saline (BBS), pH 7.4. Fresh 1% glutaraldehyde in BBS was prepared and added to the peptide to a final 5 mM glutaraldehyde concentration and incubated in an Eppendorf block at 800 rpm at 56° C. for 16 hrs. The solution was then quenched with 0.5 M glycine to make the solution 100 mM in glycine. After five minutes the solution was diluted 1:3 with BBS, dialyzed extensively against 200 volumes and three changes of 2 mM BBS overnight at 4° C., aliquoted, and lyophilized.

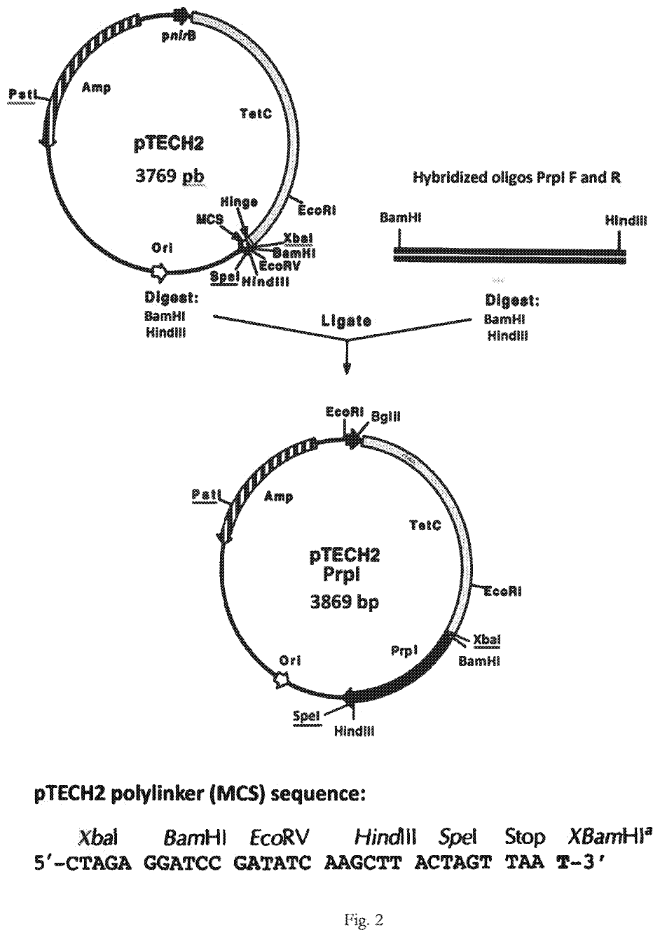

Plasmid Construction. Expression plasmid pTECH2 and hybridized oligonucleotide pairs (forward and reverse), having the nucleic acid sequences shown in Table 2, were each digested with BamHI and HindIII and then ligated together to form the expression vectors pTECH2-PrP I, -PrP II, -PrP III, -PrP IV, -PrP V, and -PrP VI, containing a single copy of the cloned peptide (x1), as shown in FIG. 2 for pTECH2-PrP I. BamHI recognition sequences are shown in bold underline; HindIII recognition sequences are shown in italic underline. The HindIII recognition in the pTECH2 polylinker (MCS) sequence (5′-CTAGA GGATCC GATATC, AAGCTT, ACTAGT, TAA T-3′ (SEQ ID NO:70), as shown in FIG. 2) was eliminated.

For construction of plasmids expressing tandem copies of the cloned peptides (x2), unique PstI, XbaI, and SbeI sites were used (FIG. 2, underlined).

TABLE 2
Nucleotide Sequences for Cloning

| Peptide | | | SEQ ID |
|---|---|---|---|
| Name | | Nucleotide Sequence for Cloning (5′-3′) | NO: |
| Prp I | Forward | GCG [symbol] CGTTACCCTCCGCAGGGCGGTGGCT | 71 |
| | | GGGGCCAGCCGCACGGTGGCGGTTGGGGCCAACC | |
| | | GCATGGCGGTGGTAGCGGCAGCGGTAAG | |
| | | CTTGCT | |
| | Reverse | AGCAAGCTTACCGCTGCCGCTACCACCGCCATGCG | 72 |
| | | GTTGGCCCCAACCGCCACCGTGCGGCTGGCCCCA | |
| | | GCCACCGCCCTGCGGAGGGTAACG [symbol] | |
| | | [symbol] CGC | |
| Prp II | Forward | GCG [symbol] CAGGGTGGCAGCCATAGCCAATGG | 73 |
| | | AATAAACCGTCCAAGCCGAAAACGAACCTGAAAC | |
| | | ACATGGCCGGCGCCGCGGCCGCGGGTGCG | |
| | | GTGAAGCTTGCT | |
| | Reverse | AGCAAGCTTCACCGCACCCGCGGCCGCGGCGCCG | 74 |
| | | GCCATGTGTTTCAGGTTCGTTTTCGGCTTGGACGG | |
| | | TTTATTCCATTGGCTATGGCTGCCACC | |
| | | CTG [symbol] CGC | |
| Prp III | Forward | GCG [symbol] GGCGCCGTGGTCGGTGGCCTGGGTG | 75 |
| | | GCTATATGCTGGGTAGCGCCATGTCCCGTCCGATT | |
| | | ATCCATTTTGGTAATGACTGGGAAGGT | |
| | | AGCGGCAAGCTTGCT | |
| | Reverse | AGCAAGCTTGCCGCTACCTTCCCAGTCATTACCAA | 76 |
| | | AATGGATAATCGGACGGGACATGGCGCTACCCAG | |
| | | CATATAGCCACCCAGGCCACCGACCAC | |
| | | GGCGCC [symbol] CGC | |
| Prp IV | Forward | GCG [symbol] GGTAGCGATTACGAAGATCGTTATT | 77 |
| | | ACCGCGAGAATATGTATCGTTACCCGGAGCAGGT | |
| | | CTATTACCGCCCGATGGACCGCTATGGT | |
| | | AGCGGCAAGCTTGCT | |
| | Reverse | AGCAAGCTTGCCGCTACCATAGCGGTCCATCGGGC | 78 |
| | | GGTAATAGACCTGCTCCGGGTAACGATACATATT | |
| | | CTCGCGGTAATAACGATCTTCGTAATC | |
| | | GCTACC [symbol] CGC | |
| Prp V | Forward | GCG [symbol] TATCGTCCGATGGATGAATACTCCA | 79 |
| | | GCCAGAATTCCTTCGTGCGTGATTGTGTCAACATC | |
| | | ACCATTAAACAGCACACCGTCACCACG | |
| | | AAGCTTGCT | |
| | Reverse | AGCAAGCTTCGTGGTGACGGTGTGCTGTTTAATGG | 80 |
| | | TGATGTTGACACAATCACGCACGAAGGAATTCTG | |
| | | GCTGGAGTATTCATCCATCGGACGATA | |
| | | [symbol] CGC | |
| Prp VI | Forward | GCG [symbol] ACCACGACCACGAAAGGCGAGAAC | 81 |
| | | TTCACCGAAACGGATATCAAAATGATGGAACGTG | |
| | | TGGTCGAACAGATGTGTGTGACGCAGTAT | |
| | | GAGCGTAAGCTTGCT | |
| | Reverse | AGCAAGCTTACGCTCATACTGCGTCACACACATCT | 82 |
| | | GTTCGACCACACGTTCCATCATTTTGATATCCGTT | |
| | | TCGGTGAAGTTCTCGCCTTTCGTGGT | |
| | | CGTGGT [symbol] CGC | |

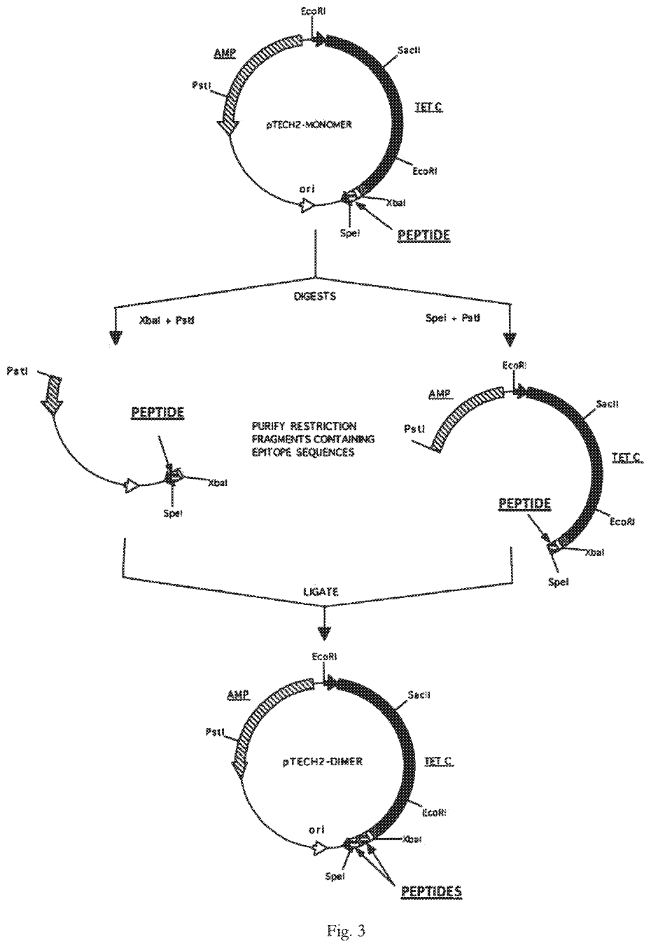

Recombinant fusion vectors expressing a single copy (x1) or two tandem copies (x2) of the PrP I, -II, -II, -IV, -V, and -VI peptides as C-terminal fusion to tetanus toxin fragment C in a live aroC attenuated vaccine strain of Salmonella LVR01 were created (as shown in FIG. 3 for tandem copies), using a modification of the method described in Khan et al., “Construction, Expression, and Immunogenicity of Multiple Tandem Copies of the Schistosoma mansoni Peptide 115-131 of the P28 Glutathione S-transferase Expressed as C-terminal fusions to Tetanus Toxin Fragment C in a Live aro-Attenuated Vaccine Strain of Salmonella,” J. Immunol. 153(12):5634-42 (1994), which is hereby incorporated by reference in its entirety.

### Example 1—Live Attenuated Vaccine Strains of Salmonella LVR01 Expressing Multi-Species Peptides

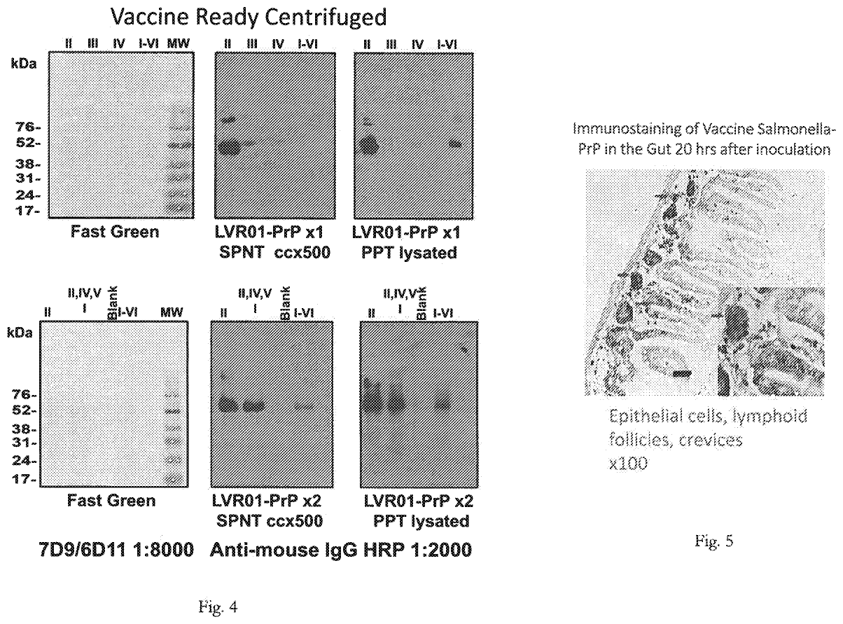

Prp multi-species peptides I, II, III, IV, V, and VI (as described supra) were expressed in attenuated Salmonella LVR01 (as described supra). Immunoblotting showed the presence of multi-species PrP peptides I-VI in the supernantants of the vaccine ready corresponding Salmonellas and the expression shown in the lysates of the same vaccine ready Salmonellas (FIG. 4). The anti-PrP antibodies used (monoclonal antibodies 7D9 and 6D11) do not recognize epitopes along the entire PrP molecule, which resulted in differential detection of different peptides. All Salmonella vaccines showed excellent multi-species PrP peptide production and secretion to the medium, with no decrease by competition when groups of three of the six or all six were grown together (FIG. 4).

The vaccine ready Salmonella LVR01 multi-species PrP I-VI were kept for 7 days at 4° C. and at room temperature (RT) for 36 hours before inoculation. The viability of the single or combined vaccines, measured as the percentage of bacteria determined by light microscopy in a Neubauer camera that showed some Brownian movement within 2 minutes of the observation; was between 87 and 95%, which makes all preparations suitable for scale production and for use combined with food in confined environments or as bait in the open field.

Antisera against Salmonella show the vaccine attenuated bacteria to be located in lymphoid follicles of the crevices and in epithelial cells of a mouse inoculated with the attenuated Salmonella vaccine 20 hours prior (FIG. 5). This argues in favor of the efficacy of delivery by the Salmonella vaccines to the specific immune presenting cells in the gut.

Multi-species peptides I-VI were expressed in vaccine attenuated Salmonella LVR01 either as a single copy (x1) or two tandem copies (x2), or synthesized and control polymerized with glutaraldehyde. Transgenic mice were inoculated weekly three times, twice with the Salmonella vaccine and the last one with the corresponding control polymerized synthetic peptides in sterile saline and alum (Al(OH)₃), as described in Goni et al., “Mucosal Immunization With an Attenuated Salmonella Vaccine Partially Protects White-Tailed Deer From Chronic Wasting Disease,” Vaccine 33:726-33 (2015), which is hereby incorporated by reference in its entirety. Transgenic mice included KO PrP (knock out for PrP), Hu PRP (transgenic for human PrP), Sh PrP (transgenic for sheep PrP), Elk PrP (transgenic for Elk-Deer PrP), and All PrP (including the wild type mouse background PrP). Control animals were inoculated with the same attenuated Salmonella LVR01 carrying the TETc fragment alone and the last boost with sterile saline-alum vehicle alone.

Immunoblots of different prion disease samples (human, deer-CWD, sheep-scrape) were run on 12.5% SDS-PAGE gel and transferred onto nitrocellulose and incubated at room temperature for one hour with mouse plasma diluted 1:1000 in TBS-T, from samples obtained from the corresponding animals 10 days after the third inoculation (second boost). The secondary one hour incubation was performed with antisera anti-mouse GAM (IgG-IgA-IgM) conjugated to horse radish peroxidase. The blots were developed by ECL and recorded on X-ray films (FIG. 6A). All combinations reacted with all different PrP species and with oligomeric forms of the different prions (FIG. 6A). The LVR01(−) acted as a negative background control and the corresponding blots were over-exposed at least 5 times longer than the rest of the positive samples to ensure the background reading of the secondary antibodies and the lack of primary antibodies to PrP sequences (FIGS. 6A, 6B).

At the same time, samples from feces of the corresponding animals were collected, homogenized and diluted 1:1000 to be used as primary antibody on similar blots; the secondary antibody was specific anti-mouse IgA-HRP to detect the most important antibody that could be produced in the intestinal mucosa (FIG. 6B). The results indicate that all intestinal antibodies developed by the different multi-species combination inoculations reacted with all species of actual prion PrP from different species prionic diseases.

All methodologies used were similar to the ones detailed in Goni et al., “High Titers of Mucosal and Sytemic anti-PRP Antibodies Abrogate Oral Prion Infection in Mucosal-Vaccinated Mice,” Neuroscience 153(3):679-86 (2008).

What is claimed is:

1. An isolated protein or peptide thereof comprising the amino acid sequence of
(SEQ ID NO: 1)
MX₂X₃X₄X₅X₆GX₈WX₁₀LX₁₂LFVX₁₆X₁₇WX₁₉DX₂₁GX₂₃CKKX₂₇
PKPGGX₃₃WNTGGX₃₉SRYPGQGX₄₇PGGNRYPX₅₅QGGX₅₉X₆₀
WGQPHGGGWGQPHGGX₇₆WGQPHGGX₈₄WGQPHGGX₉₂X₉₃X₉₄
X₉₅X₉₆X₉₇X₉₈X₉₉X₁₀₀GWGQX₁₀₅GX₁₀₇X₁₀₈HX₁₁₀QWX₁₁₃KPX₁₁₆
KPKTX₁₂₁X₁₂₂KHX₁₂₅AGAAAAGAVVGGLGGYX₁₄₂LGSAMSRP
X₁₅₁IHFGX₁₅₆DX₁₅₈EDRYYRENMX₁₆₈RYPX₁₇₂QVYYX₁₇₇PX₁₇₉
X₁₈₀X₁₈₁YX₁₈₃X₁₈₄QNX₁₈₇FVX₁₉₀DCVNITX₁₉₇KX₁₉₉HTVTTTT
KGENFTETDX₂₁₆KX₂₁₈ME X₂₂₁VVEQMCX₂₂₈TQYX₂₃₂X₂₃₃ EX₂₃₅
X₂₃₆ AX₂₃₈X₂₃₉ X₂₄₀ X₂₄₁ X₂₄₂ X₂₄₃ X₂₄₄ X₂₄₅ X₂₄₆ X₂₄₇
X₂₄₈ X₂₄₉ FSX₂₅₂PPVX₂₅₆LLISX₂₆₁LIX₂₆₄ LIVX₂₆₈
wherein X₂ is V or A; X₃ is K or null; X₄ is S or null; X₅ is N or H; X₆ is I, V, L or M; X₈ is S, G, Y or C; X₁₀ is I, L or M; X₁₂ is V, A, or L; X₁₆ is A, T, or V; X₁₇ is T or M; X₁₉ is S or T; X₂₁ is V, I, M; X₂₃ is L or F; X₂₇ is R or W; X₃₃ is G or null; X₃₉ is G or null; X₄₇ S or I; X₅₅ is P or S; X₅₉ is G or null; X₆₀ is G or T; X₇₆ is G or S; X₈₄ is G or S; X₉₂ is G or null; X₉₃ is W or null; X₉₄ is G or null; X₉₅ is Q or null; X₉₆ is P or null; X₉₇ is H or null; X₉₈ is G or null; X₉₉ is G or null; X₁₀₀ is G or null; X₁₀₅ is G or S; X₁₀₇ is G or null; X₁₀₈ is T, A, or S; X₁₁₀ is G, N, or S; X₁₁₃ is N or G; X₁₁₆ is S or N; X₁₂₁ is N or S; X₁₂₂ is M or L; X₁₂₅ is V or M; X₁₄₂ is M or L; X₁₅₁ is L, M or I; X₁₅₆ is N or S; X₁₅₈ is Y or W; X₁₆₈ is Y or H; X₁₇₂ is N or E; X₁₇₇ is R or K; X₁₇₉ is V or M; X₁₈₀ is D, N or S; X₁₈₁ is Q, R or E; X₁₈₃ is S or N; X₁₈₄ is N or S; X₁₈₇ is N, S, or T, X₁₉₀ is H or R; X₁₉₇ is V or I; X₁₉₉ is Q or E; X₂₁₆ is V, M or I; X₂₁₈ is M or I; X₂₂₁ is R or Q; X₂₂₈ is I or V; X₂₃₂ is Q, E, or R; X₂₃₃ R, Q, or K; X₂₃₅ is S or Y; X₂₃₆ is Q or E; X₂₃₈ is Y, S, F, or A; X₂₃₉ is Y or Q; X₂₄₀ is Q, D, or G; X₂₄₁ is G or null; X₂₄₂ R or null; X₂₄₃ is R or K; X₂₄₄ is G, S, or A; X₂₄₅ is A or S; X₂₄₆ is S or G; X₂₄₇ is V, T, A, M or null; X₂₄₈ is I, L, V; X₂₄₉ is L or I; X₂₅₂ is S or P; X₂₅₆ is I or V; X₂₆₁ is F or L; X₂₆₄ is F or L; X₂₆₈ is G or R; and
wherein the isolated protein or peptide thereof does not comprise an amino acid sequence of a corresponding protein of any one of SEQ ID Nos: 2-34 or a corresponding peptide thereof.

2. The isolated peptide of claim 1, wherein said peptide comprises the amino acid sequence of amino acid residues 52-75 of SEQ ID NO: 1
(RYPX₅₅QGGX₅₉X₆₀WGQPHGGGWGQPHGG (SEQ ID NO: 35)).

3. The isolated peptide of claim 1, wherein said peptide comprises the amino acid sequence of amino acid residues 104-134 of SEQ ID NO: 1
(QX₁₀₅GX₁₀₇X₁₀₈HX₁₁₀QWX₁₁₃KPX₁₁₆KPKTX₁₂₁X₁₂₂KHX₁₂₅AGAAAAGAV
(SEQ ID NO: 36)).

4. The isolated peptide of claim 1, wherein said peptide comprises the amino acid sequence of amino acid residues 155-182 of SEQ ID NO: 1 (GX₁₅₆DX₁₅₈EDRYYREN MX₁₆₈RYPX₁₇₂QVYYX₁₇₇PX₁₇₉X₁₈₀X₁₈₁Y (SEQ ID NO: 37)).

5. The isolated peptide of claim 1, wherein said peptide comprises the amino acid sequence of amino acid residues 132-159 of SEQ ID NO: 1 (GAVVGGLGGYX₁₄₂L GSAMSRP X₁₅₁IHFGX₁₅₆DX₁₅₈E (SEQ ID NO: 38)).

6. The isolated peptide of claim 1, wherein said peptide comprises the amino acid sequence of amino acid residues 203-233 of SEQ ID NO: 1 (TTTTKGENFTETD X₂₁₆KX₂₁₈ME X₂₂₁VVEQMCX₂₂₈TQYX₂₃₂X₂₃₃ (SEQ ID NO: 39)).

7. The isolated peptide of claim 1, wherein said peptide comprises the amino acid sequence of amino acid residues 176-204 of SEQ ID NO: 1 (YX₁₇₇PX₁₇₉X₁₈₀X₁₈₁YX₁₈₃ X₁₈₄QNX₁₈₇ FVX₁₉₀DCVNITX₁₉₇KX₁₉₉HTVTT (SEQ ID NO: 40)).

8. The isolated protein or peptide thereof of any one of claims 1-7, wherein said protein or peptide thereof comprises an amino acid sequence having at least two single amino acid residue variations relative to the amino acid sequence of a corresponding protein of any one of SEQ ID Nos: 2-34 or peptide thereof.

9. The isolated protein or peptide thereof of any one of claims 1-7, wherein said protein or peptide thereof comprises an amino acid sequence having at least three single amino acid variations relative to the amino acid sequence of a corresponding protein of any one of SEQ ID Nos: 2-34 or peptide thereof.

10. The isolated protein or peptide thereof of any one of claims 1-9, wherein said peptide is at least 15 amino acid residues in length.

11. The isolated protein or peptide thereof of any one of claims 1-9, wherein said peptide is at least 25 amino acid residues in length.

12. The isolated protein or peptide thereof of any one of claims 1-9, wherein said peptide is at least 35 amino acid residues in length.

13. The isolated protein or peptide thereof of any one of claims 1-12, wherein said peptide is no more than 150 amino acid residues in length.

14. The isolated protein or peptide thereof of any one of claims 1-12, wherein said peptide is no more than 100 amino acid residues in length.

15. The isolated protein or peptide thereof of any one of claims 1-12, wherein said peptide is no more than 75 amino acid residues in length.

16. The isolated protein or peptide thereof of any one of claims 1-15 further comprising:
an adjuvant polypeptide linked in frame to said protein or peptide thereof.

17. The isolated protein or peptide thereof of claim 16, wherein the adjuvant polypeptide is selected from the group consisting of cholera toxin B, flagellin, human papillomavirus L1 or L2 protein, herpes simplex glycoprotein D (gD), complement C4 binding protein, TL4 ligand, influenza HA, Gb3 and 1L-1β.

18. The isolated protein or peptide thereof of claim 16 or claim 17, wherein a linker sequence couples said protein or peptide thereof to the adjuvant polypeptide.

19. The isolated protein or peptide thereof of any one of claims 1-18, wherein said isolated protein or peptide thereof further comprises a GSG adapter sequence at its amino terminus, at its carboxy terminus, or both termini.

20. The isolated protein or peptide thereof of any one of claims 1-18, wherein said protein or peptide thereof further comprises a GKSG adapter sequence at its amino terminus, a GSKG adapter sequence at its carboxy terminus, or the corresponding ones at both termini.

21. A polymer of one or more peptides as defined in any one of claims 1-20.

22. The polymer of claim 21, wherein the one or more peptides are selected from the peptides of SEQ ID NOs: 35-40;

23. The polymer of claim 21 or claim 22, wherein the polymer is a homopolymer of a single peptide.

24. The polymer of claim 21 or claim 22, wherein the polymer is a heteropolymer of two different peptides.

25. The polymer of any one of claims 21-24, wherein each peptide of the polymer is covalently linked to a another peptide of the polymer by a glutaraldehyde molecule.

26. A vaccine composition comprising:
one or more polymers of any one of claims 21-25, and
a pharmaceutically acceptable carrier.

27. The vaccine composition of claim 26, wherein said composition comprises two or more different polymers.

28. The vaccine composition of claim 26 or claim 27, wherein said composition further comprises an adjuvant.

29. The vaccine composition of claim 28, where in the adjuvant is selected from the group consisting of aluminum salt, cholera toxin subunit B, heat labile enterotoxin, flagellin, Freund's complete adjuvant, Freund's incomplete adjuvant, lysolecithin, pluronic polyols, polyanions, an oil-water emulsion, dinitrophenol, iscomatrix, influenza HA, Gb3 and liposome polycation DNA particles.

30. The vaccine composition of claim 29, wherein the adjuvant is aluminum salt.

31. A delivery vehicle comprising the vaccine composition of any one of claims 26-30.

32. The delivery vehicle of claim 31, wherein said delivery vehicle is selected from biodegradable microspheres, microparticles, nanoparticles, liposomes, micelles, collagen minipellets, and cochleates.

33. An attenuated bacterium transformed with a vector expressing one or more proteins or peptides thereof of any one of claims 1-20.

34. The attenuated bacterium of claim 33, wherein each of said proteins or peptides thereof is expressed as single units or tandemly repeated units.

35. The attenuated bacterium of claim 33 or 34, wherein the attenuated bacterium is a Salmonella strain.

36. The attenuated bacterium of claim 35, wherein the Salmonella strain is selected from the group consisting of Salmonella typhimurium LVR01 and SL3261, and Salmonella typhi Ty21a.

37. The attenuated bacterium of any one of claims 33-36, wherein the vector expresses one or more peptides selected from the peptides of SEQ ID NOs: 35-40.

38. A vaccine composition comprising:
one or more attenuated bacterium of any one of claims 33-37, and
a pharmaceutically acceptable carrier.

39. The vaccine composition of claim 38, wherein said composition comprises two or more attenuated bacterium, each attenuated bacterium transformed with a vector expressing a different protein or peptide thereof.

40. The vaccine composition of claim 39, wherein the different proteins or peptides thereof are selected from peptides of SEQ ID Nos: 35-40.

41. The vaccine composition of any one of claims 38-41, wherein said composition further comprises an adjuvant.

42. The vaccine composition of claim 41, where in the adjuvant is selected from the group consisting of aluminum hydroxide, cholera toxin subunit B, and heat labile enterotoxin.

43. The vaccine composition of claim 42, wherein the adjuvant is aluminum hydroxide.

44. A method of inhibiting the onset of or treat an existing prion disease in a mammalian subject, said method comprising:
administering, to a mammalian subject in need thereof, a vaccine composition of any one of claims 26-30 and 38-43 or the delivery vehicle of claims 31 or 32, in an amount effective to inhibit the onset of prion disease is said mammalian subject.

45. The method of claim 44, wherein said administering comprises mucosal administration.

46. The method of claim 45, wherein said mucosal administration is selected from oral, intragastric, intranasal, rectal, and intraocular mucosal administration.

47. The method of any one of claims 44-46, wherein the mammalian subject is a cow, deer, elk, sheep, buffalo, moose, bison, camel, dromedary, vicuña, alpaca, llama or guanaco.

48. The method of any one of claims 44-47, wherein the prion disease is bovine spongiform encephalopathy, chronic wasting disease, scrapie, feline spongiform encephalopathy, transmissible mink encephalopathy, exotic ungulate encephalopathy, or any form of camelid spongiform encephalopathy.

49. The method of any one of claims 44-46, wherein the mammalian subject is a human, and the prion disease is selected from sporadic or familial Creutzfeldt-Jakob disease, variant Creutzfeldt-Jakob disease, Gerstmann-Straussler-Scheinker Syndrome, Fatal Familial Insomnia, and Kuru.

50. The method of any one of claims 44-49 further comprising:
repeating said administering one or more times.

51. The method of any one of claims 44-49, wherein said administering comprises:
administering a first vaccine composition to the subject, and
boosting the subject's response by administering a second vaccine composition, wherein the second vaccine composition is different from the first vaccine composition.

52. The method of any one of claims 44-49, wherein said administering comprises:
administering a first vaccine composition to the subject, wherein said first vaccine comprises two or more attenuated bacterium, each attenuated bacterium transformed with a vector expressing a different protein or peptide thereof, and
boosting the subject's response by administering a second vaccine composition wherein the second vaccine composition is the same as the first vaccine composition.

53. An animal bait or feed comprising the vaccine composition of any one of claims 26-30 and 38-43.

54. An isolated polynucleotide encoding the isolated protein or peptide thereof of claim 1.

55. The isolated polynucleotide of claim 54, wherein said polynucleotide encodes the isolated peptide of SEQ ID NO: 35.

56. The isolated polynucleotide of claim 55, wherein said polynucleotide comprises the nucleotide sequence of SEQ ID NO: 41.

57. The isolated polynucleotide of claim 54, wherein said polynucleotide encodes the isolated peptide of SEQ ID NO: 36.

58. The isolated polynucleotide of claim 57, wherein said polynucleotide comprises the nucleotide sequence of SEQ ID NO: 42.

59. The isolated polynucleotide of claim 54, wherein said polynucleotide encodes the isolated peptide of SEQ ID NO: 37.

60. The isolated polynucleotide of claim 59, wherein said polynucleotide comprises the nucleotide sequence of SEQ ID NO: 43.

61. The isolated polynucleotide of claim 54, wherein said polynucleotide encodes the isolated peptide of SEQ ID NO: 38.

62. The isolated polynucleotide of claim 61, wherein said polynucleotide comprises the nucleotide sequence of SEQ ID NO: 44.

63. The isolated polynucleotide of claim 54, wherein said polynucleotide encodes the isolated peptide of SEQ ID NO: 39.

64. The isolated polynucleotide of claim 64, wherein said polynucleotide comprises the nucleotide sequence of SEQ ID NO: 45.

65. The isolated polynucleotide of claim 54, wherein said polynucleotide encodes the isolated peptide of SEQ ID NO: 40.

66. The isolated polynucleotide of claim 65, wherein said polynucleotide comprises the nucleotide sequence of SEQ ID NO: 46.

67. A recombinant vector comprising the isolated polynucleotide of any one of claims 54-66.

68. A host cell comprising the recombinant vector of claim 67.

69. The host cell of claim 68, wherein said host cell is an attenuated bacterial cell.

70. The host cell of claim 68, wherein the host cell is an attenuated Salmonella cell.

71. An isolated antibody or epitope-binding portion thereof, wherein said antibody or epitope-binding portion thereof binds to an epitope of a protein or peptide thereof comprising the amino acid sequence of:
(SEQ ID NO: 1)
MX₂X₃X₄X₅X₆GX₈WX₁₀LX₁₂LFVX₁₆X₁₇WX₁₉DX₂₁GX₂₃CKKX₂₇
PKPGGX₃₃WNTGGX₃₉SRYPGQGX₄₇PGGNRYPX₅₅QGGX₅₉X₆₀
WGQPHGGGWGQPHGGX₇₆WGQPHGGX₈₄WGQPHGGX₉₂X₉₃X₉₄
X₉₅X₉₆X₉₇X₉₈X₉₉X₁₀₀GWGQX₁₀₅GX₁₀₇X₁₀₈HX₁₁₀QWX₁₁₃KPX₁₁₆
KPKTX₁₂₁X₁₂₂KHX₁₂₅AGAAAAGAVVGGLGGYX₁₄₂LGSAMSRP
X₁₅₁IHFGX₁₅₆DX₁₅₈EDRYYRENMX₁₆₈RYPX₁₇₂QVYYX₁₇₇PX₁₇₉
X₁₈₀X₁₈₁YX₁₈₃X₁₈₄QNX₁₈₇FVX₁₉₀DCVNITX₁₉₇KX₁₉₉HTVTTTT
KGENFTETDX₂₁₆KX₂₁₈ME X₂₂₁VVEQMCX₂₂₈TQYX₂₃₂X₂₃₃ EX₂₃₅
X₂₃₆ AX₂₃₈X₂₃₉ X₂₄₀ X₂₄₁ X₂₄₂ X₂₄₃ X₂₄₄ X₂₄₅ X₂₄₆ X₂₄₇
X₂₄₈ X₂₄₉ FSX₂₅₂PPVX₂₅₆LLISX₂₆₁LIX₂₆₄ LIVX₂₆₈
wherein X₂ is V or A; X₃ is K or null; X₄ is S or null; X₅ is N or H; X₆ is I, V, L or M; X₈ is S, G, Y or C; X₁₀ is I, L or M; X₁₂ is V, A, or L; X₁₆ is A, T, or V; X₁₇ is T or M; X₁₉ is S or T; X₂₁ is V, I, M; X₂₃ is L or F; X₂₇ is R or W; X₃₃ is G or null; X₃₉ is G or null; X₄₇ S or I; X₅₅ is P or S; X₅₉ is G or null; X₆₀ is G or T; X₇₆ is G or S; X₈₄ is G or S; X₉₂ is G or null; X₉₃ is W or null; X₉₄ is G or null; X₉₅ is Q or null; X₉₆ is P or null; X₉₇ is H or null; X₉₅ is G or null; X₉₉ is G or null; X₁₀₀ is G or null; X₁₀₅ is G or S; X₁₀₇ is G or null; X₁₀₈ is T, A, or S; X₁₁₀ is G, N, or S; X₁₁₃ is N or G; X₁₁₆ is S or N; X₁₂₁ is N or S; X₁₂₂ is M or L; X₁₂₅ is V or M; X₁₄₂ is M or L; X₁₅₁ is L, M or I; X₁₅₆ is N or S; X₁₅₈ is Y or W; X₁₆₈ is Y or H; X₁₇₂ is N or E; X₁₇₇ is R or K; X₁₇₉ is V or M; X₁₈₀ is D, N or S; X₁₈₁ is Q, R or E; X₁₈₃ is S or N; X₁₈₄ is N or S; X₁₈₇ is N, S, or T, X₁₉₀ is H or R; X₁₉₇ is V or I; X₁₉₉ is Q or E; X₂₁₆ is V, M or I; X₂₁₈ is M or I; X₂₂₁ is R or Q; X₂₂₈ is I or V; X₂₃₂ is Q, E, or R; X₂₃₃ R, Q, or K; X₂₃₅ is S or Y; X₂₃₆ is Q or E; X₂₃₈ is Y, S, F, or A; X₂₃₉ is Y or Q; X₂₄₀ is Q, D, or G; X₂₄₁ is G or null; X₂₄₂ R or null; X₂₄₃ is R or K; X₂₄₄ is G, S, or A; X₂₄₅ is A or S; X₂₄₆ is S or G; X₂₄₇ is V, T, A, M or null; X₂₄₈ is I, L, V; X₂₄₉ is L or I; X₂₅₂ is S or P; X₂₃₆ is I or V; X₂₆₁ is F or L; X₂₆₄ is F or L; X₂₆₈ is G or R; and
wherein the protein or peptide thereof does not comprise an amino acid sequence of a corresponding protein of any one of SEQ ID Nos: 2-34 or a corresponding peptide thereof.

72. The antibody or epitope-binding fragment thereof of claim 71, wherein said antibody was raised against the protein or a peptide thereof of SEQ ID NO: 1.

73. An isolated antibody or epitope-binding portion thereof, wherein said antibody or epitope-binding portion thereof binds to a polymer of a protein or peptide thereof, wherein said protein or peptide thereof of the polymer comprises the amino acid sequence of:
(SEQ ID NO: 1)
MX₂X₃X₄X₅X₆GX₈WX₁₀LX₁₂LFVX₁₆X₁₇WX₁₉DX₂₁GX₂₃CKKX₂₇
PKPGGX₃₃WNTGGX₃₉SRYPGQGX₄₇PGGNRYPX₅₅QGGX₅₉X₆₀
WGQPHGGGWGQPHGGX₇₆WGQPHGGX₈₄WGQPHGGX₉₂X₉₃X₉₄
X₉₅X₉₆X₉₇X₉₈X₉₉X₁₀₀GWGQX₁₀₅GX₁₀₇X₁₀₈HX₁₁₀QWX₁₁₃KPX₁₁₆
KPKTX₁₂₁X₁₂₂KHX₁₂₅AGAAAAGAVVGGLGGYX₁₄₂LGSAMSRP
X₁₅₁IHFGX₁₅₆DX₁₅₈EDRYYRENMX₁₆₈RYPX₁₇₂QVYYX₁₇₇PX₁₇₉
X₁₈₀X₁₈₁YX₁₈₃X₁₈₄QNX₁₈₇FVX₁₉₀DCVNITX₁₉₇KX₁₉₉HTVTTTT
KGENFTETDX₂₁₆KX₂₁₈ME X₂₂₁VVEQMCX₂₂₈TQYX₂₃₂X₂₃₃ EX₂₃₅
X₂₃₆ AX₂₃₈X₂₃₉ X₂₄₀ X₂₄₁ X₂₄₂ X₂₄₃ X₂₄₄ X₂₄₅ X₂₄₆ X₂₄₇
X₂₄₈ X₂₄₉ FSX₂₅₂PPVX₂₅₆LLISX₂₆₁LIX₂₆₄ LIVX₂₆₈
wherein X₂ is V or A; X₃ is K or null; X₄ is S or null; X₅ is N or H; X₆ is I, V, L or M; X₈ is S, G, Y or C; X₁₀ is I, L or M; X₁₂ is V, A, or L; X₁₆ is A, T, or V; X₁₇ is T or M; X₁₉ is S or T; X₂₁ is V, I, M; X₂₃ is L or F; X₂₇ is R or W; X₃₃ is G or null; X₃₉ is G or null; X₄₇ S or I; X₅₅ is P or S; X₅₉ is G or null; X₆₀ is G or T; X₇₆ is G or S; X₈₄ is G or S; X₉₂ is G or null; X₉₃ is W or null; X₉₄ is G or null; X₉ is Q or null; X₉₆ is P or null; X₉₇ is H or null; X₉₈ is G or null; X₉₉ is G or null; X₁₀₀ is G or null; X₁₀₅ is G or S; X₁₀₇ is G or null; X₁₀₈ is T, A, or S; X₁₁₀ is G, N, or S; X₁₁₃ is N or G; X₁₁₆ is S or N; X₁₂₁ is N or S; X₁₂₂ is M or L; X₁₂₅ is V or M; X₁₄₂ is M or L; X₁₅₁ is L, M or I; X₁₅₆ is N or S; X₁₅₈ is Y or W; X₁₆₈ is Y or H; X₁₇₂ is N or E; X₁₇₇ is R or K; X₁₇₉ is V or M; X₁₈₀ is D, N or S; X₁₈₁ is Q, R or E; X₁₈₃ is S or N; X₁₈₄ is N or S; X₁₈₇ is N, S, or T, X₁₉₀ is H or R; X₁₉₇ is V or I; X₁₉₉ is Q or E; X₂₁₆ is V, M or I; X₂₁₈ is M or I; X₂₂₁ is R or Q; X₂₂₈ is I or V; X₂₃₂ is Q, E, or R; X₂₃₃ R, Q, or K; X₂₃₅ is S or Y; X₂₃₆ is Q or E; X₂₃₈ is Y, S, F, or A; X₂₃₉ is Y or Q; X₂₄₀ is Q, D, or G; X₂₄₁ is G or null; X₂₄₂ R or null; X₂₄₃ is R or K; X₂₄₄ is G, S, or A; X₂₄₅ is A or S; X₂₄₆ is S or G; X₂₄₇ is V, T, A, M or null; X₂₄₈ is I, L, V; X₂₄₉ is L or I; X₂₅₂ is S or P; X₂₃₆ is I or V; X₂₆₁ is F or L; X₂₆₄ is F or L; X₂₆₈ is G or R; and
wherein the protein or peptide thereof does not comprise an amino acid sequence of a corresponding protein of any one of SEQ ID Nos: 2-34 or a corresponding peptide thereof.

74. The antibody or epitope-binding fragment thereof of claim 73, wherein said antibody was raised against said polymer.

75. A method of inhibiting the onset of or treating a prion disease in a mammalian subject, said method comprising:
administering, to the subject, the antibody or epitope-binding portion thereof of any one of claims 71-74 in an amount effective to inhibit the onset of or treat the prion disease in the mammalian subject.

76. The method of claim 75, wherein the mammalian subject is a cow, deer, elk, sheep, buffalo, moose, bison, camel, dromedary, vicuña, alpaca, llama or guanaco.

77. The method of 75, wherein the prion disease is bovine spongiform encephalopathy, chronic wasting disease, scrapie, feline spongiform encephalopathy, transmissible mink encephalopathy, exotic ungulate encephalopathy, or any form of camelid spongiform encephalopathy.

78. The method of 75, wherein the mammalian subject is a human, and the prion disease is selected from sporadic or familial Creutzfeldt-Jakob disease, variant Creutzfeldt-Jakob disease, Gerstmann-Straussler-Scheinker Syndrome, Fatal Familial Insomnia, and Kuru.

79. The method of any one of claims 75-78 further comprising:
repeating said administering one or more times.
